SEQUENCE LISTING (74 sequences)

SEQ ID NO: 1 - DNA, 20 bp, Artificial Sequence
  misc_feature: Synthetic Oligonucleotide
ggggtcaacgttcagggggg

SEQ ID NO: 2 - DNA, 15 bp, Artificial Sequence
  misc_feature: Synthetic Oligonucleotide
gctagacgttagcgt

SEQ ID NO: 3 - DNA, 15 bp, Artificial Sequence
  misc_feature: Synthetic Oligonucleotide
gctagatgttagcgt

SEQ ID NO: 4 - DNA, 15 bp, Artificial Sequence
  misc_feature: Synthetic Oligonucleotide
gctagangttagcgt

SEQ ID NO: 5 - DNA, 15 bp, Artificial Sequence
  misc_feature: Synthetic Oligonucleotide
gctagacgttagngt

SEQ ID NO: 6 - DNA, 15 bp, Artificial Sequence
  misc_feature: Synthetic Oligonucleotide
gcatgacgttgagct

SEQ ID NO: 7 - DNA, 20 bp, Artificial Sequence
  misc_feature: Synthetic Oligonucleotide
atggaaggtccagcgttctc

SEQ ID NO: 8 - DNA, 20 bp, Artificial Sequence
  misc_feature: Synthetic Oligonucleotide
atcgactctcgagcgttctc

SEQ ID NO: 9 - DNA, 20 bp, Artificial Sequence
  misc_feature: Synthetic Oligonucleotide
atngactctngagngttctc

SEQ ID NO: 10 - DNA, 20 bp, Artificial Sequence
  misc_feature: Synthetic Oligonucleotide
atngactctcgagcgttctc

SEQ ID NO: 11 - DNA, 20 bp, Artificial Sequence
  misc_feature: Synthetic Oligonucleotide
atcgactctcgagcgttntc

SEQ ID NO: 12 - DNA, 20 bp, Artificial Sequence
  misc_feature: Synthetic Oligonucleotide
atggaaggtccaacgttctc

SEQ ID NO: 13 - DNA, 20 bp, Artificial Sequence
  misc_feature: Synthetic Oligonucleotide
gagaacgctggaccttccat

SEQ ID NO: 14 - DNA, 20 bp, Artificial Sequence
  misc_feature: Synthetic Oligonucleotide
gagaacgctcgaccttccat

SEQ ID NO: 15 - DNA, 20 bp, Artificial Sequence
  misc_feature: Synthetic Oligonucleotide
gagaacgctcgaccttcgat

SEQ ID NO: 16 - DNA, 20 bp, Artificial Sequence
  misc_feature: Synthetic Oligonucleotide
gagcaagctggaccttccat

SEQ ID NO: 17 - DNA, 20 bp, Artificial Sequence
  misc_feature: Synthetic Oligonucleotide
gagaangctggaccttccat

SEQ ID NO: 18 - DNA, 20 bp, Artificial Sequence
  misc_feature: Synthetic Oligonucleotide
gagaacgctggacnttccat

SEQ ID NO: 19 - DNA, 20 bp, Artificial Sequence
  misc_feature: Synthetic Oligonucleotide
gagaacgatggaccttccat

SEQ ID NO: 20 - DNA, 20 bp, Artificial Sequence
  misc_feature: Synthetic Oligonucleotide
gagaacgctccagcactgat

SEQ ID NO: 21 - DNA, 20 bp, Artificial Sequence
  misc_feature: Synthetic Oligonucleotide
tccatgtcggtcctgatgct

SEQ ID NO: 22 - DNA, 20 bp, Artificial Sequence
  misc_feature: Synthetic Oligonucleotide
tccatgctggtcctgatgct

SEQ ID NO: 23 - DNA, 20 bp, Artificial Sequence
  misc_feature: Synthetic Oligonucleotide
tccatgtnggtcctgatgct

SEQ ID NO: 24 - DNA, 20 bp, Artificial Sequence
  misc_feature: Synthetic Oligonucleotide
tccatgtcggtnctgatgct

SEQ ID NO: 25 - DNA, 20 bp, Artificial Sequence
  misc_feature: Synthetic Oligonucleotide
tccatgacgttcctgatgct

SEQ ID NO: 26 - DNA, 20 bp, Artificial Sequence
  misc_feature: Synthetic Oligonucleotide
tccatgtcggtcctgctgat

SEQ ID NO: 27 - DNA, 11 bp, Artificial Sequence
  misc_feature: Synthetic Oligonucleotide
tcntnnncgnn

SEQ ID NO: 28 - DNA, 10 bp, Artificial Sequence
  misc_feature: Synthetic Oligonucleotide
tcntnngcnn

SEQ ID NO: 29 - DNA, 24 bp, Artificial Sequence
  misc_feature: Synthetic Oligonucleotide
tcgtcgttttgtcgttttgtcgtt

SEQ ID NO: 30 - DNA, 19 bp, Artificial Sequence
  misc_feature: Synthetic Oligonucleotide
ggggacgtcgacgtggggg

SEQ ID NO: 31 - DNA, 11 bp, Artificial Sequence
  misc_feature: Synthetic Oligonucleotide
tcntnnncgtt

SEQ ID NO: 32 - DNA, 18 bp, Artificial Sequence
  misc_feature: Synthetic Oligonucleotide
tcntnnncgttnnncgtt

SEQ ID NO: 33 - DNA, 25 bp, Artificial Sequence
  misc_feature: Synthetic Oligonucleotide
tcntnnncgttnnncgttnnncgtt

SEQ ID NO: 34 - DNA, 20 bp, Artificial Sequence
  misc_feature: Synthetic Oligonucleotide
gtcgttngtcgttngtcgtt

SEQ ID NO: 35 - DNA, 27 bp, Artificial Sequence
  misc_feature: Synthetic Oligonucleotide
tcgtcgttttgtcgttttgtcgttttt

SEQ ID NO: 36 - DNA, 27 bp, Artificial Sequence
  misc_feature: Synthetic Oligonucleotide
tcgtcgttttgtcgttttgtcgttccc

SEQ ID NO: 37 - DNA, 27 bp, Artificial Sequence
  misc_feature: Synthetic Oligonucleotide
tcgtcgttttgtcgttttgtcgttaaa

SEQ ID NO: 38 - DNA, 24 bp, Artificial Sequence
  misc_feature: Synthetic Oligonucleotide
tcgtcgttttgtcgttttgtcgtt

SEQ ID NO: 39 - DNA, 20 bp, Artificial Sequence
  misc_feature: Synthetic Oligonucleotide
atcgactctcgagcgttctc

SEQ ID NO: 40 - DNA, 20 bp, Artificial Sequence
  misc_feature: Synthetic Oligonucleotide
atcgactctcgagcgttntc

SEQ ID NO: 41 - DNA, 20 bp, Artificial Sequence
  misc_feature: Synthetic Oligonucleotide
tccacgacgttttcgacgtt

SEQ ID NO: 42 - DNA, 20 bp, Artificial Sequence
  misc_feature: Synthetic Oligonucleotide
tccataacgttcctgatgct

SEQ ID NO: 43 - DNA, 20 bp, Artificial Sequence
  misc_feature: Synthetic Oligonucleotide
tccatagcgttcctagcgtt

SEQ ID NO: 44 - DNA, 20 bp, Artificial Sequence
  misc_feature: Synthetic Oligonucleotide
tccatcacgtgcctgatgct

SEQ ID NO: 45 - DNA, 20 bp, Artificial Sequence
  misc_feature: Synthetic Oligonucleotide
tccatgacggtcctgatgct

SEQ ID NO: 46 - DNA, 20 bp, Artificial Sequence
  misc_feature: Synthetic Oligonucleotide
tccatgacgtccctgatgct

SEQ ID NO: 47 - DNA, 20 bp, Artificial Sequence
  misc_feature: Synthetic Oligonucleotide
tccatgacgttcctgacgtt

SEQ ID NO: 48 - DNA, 20 bp, Artificial Sequence
  misc_feature: Synthetic Oligonucleotide
tccatgacgttcctgatgct

SEQ ID NO: 49 - DNA, 20 bp, Artificial Sequence
  misc_feature: Synthetic Oligonucleotide
tccatgccggtcctgatgct

SEQ ID NO: 50 - DNA, 20 bp, Artificial Sequence
  misc_feature: Synthetic Oligonucleotide
tccatgcgttgcgttgcgtt

SEQ ID NO: 51 - DNA, 20 bp, Artificial Sequence
  misc_feature: Synthetic Oligonucleotide
tccatggcggtcctgatgct

SEQ ID NO: 52 - DNA, 20 bp, Artificial Sequence
  misc_feature: Synthetic Oligonucleotide
tccatgtcgatcctgatgct

SEQ ID NO: 53 - DNA, 20 bp, Artificial Sequence
  misc_feature: Synthetic Oligonucleotide
tccatgtcgctcctgatgct

SEQ ID NO: 54 - DNA, 20 bp, Artificial Sequence
  misc_feature: Synthetic Oligonucleotide
tccatgtcggtcctgatgct

SEQ ID NO: 55 - DNA, 20 bp, Artificial Sequence
  misc_feature: Synthetic Oligonucleotide
tccatgtcggtcctgctgat

SEQ ID NO: 56 - DNA, 20 bp, Artificial Sequence
  misc_feature: Synthetic Oligonucleotide
tccatgtcggtnctgatgct

SEQ ID NO: 57 - DNA, 20 bp, Artificial Sequence
  misc_feature: Synthetic Oligonucleotide
tccatgtcgttcctgatgct

SEQ ID NO: 58 - DNA, 20 bp, Artificial Sequence
  misc_feature: Synthetic Oligonucleotide
tccatgtcgttcctgtcgtt

SEQ ID NO: 59 - DNA, 19 bp, Artificial Sequence
  misc_feature: Synthetic Oligonucleotide
tcctgacgttcctgacgtt

SEQ ID NO: 60 - DNA, 19 bp, Artificial Sequence
  misc_feature: Synthetic Oligonucleotide
tcctgtcgttcctgtcgtt

SEQ ID NO: 61 - DNA, 20 bp, Artificial Sequence
  misc_feature: Synthetic Oligonucleotide
tcctgtcgttccttgtcgtt

SEQ ID NO: 62 - DNA, 20 bp, Artificial Sequence
  misc_feature: Synthetic Oligonucleotide
tcctgtcgttttttgtcgtt

SEQ ID NO: 63 - DNA, 20 bp, Artificial Sequence
  misc_feature: Synthetic Oligonucleotide
tccttgtcgttcctgtcgtt

SEQ ID NO: 64 - DNA, 21 bp, Artificial Sequence
  misc_feature: Synthetic Oligonucleotide
tcgtcgctgttgtcgtttctt

SEQ ID NO: 65 - DNA, 14 bp, Artificial Sequence
  misc_feature: Synthetic Oligonucleotide
tcgtcgtcgtcgtt

SEQ ID NO: 66 - DNA, 20 bp, Artificial Sequence
  misc_feature: Synthetic Oligonucleotide
tcgtcgttgtcgttgtcgtt

SEQ ID NO: 67 - DNA, 22 bp, Artificial Sequence
  misc_feature: Synthetic Oligonucleotide
tcgtcgttgtcgttttgtcgtt

SEQ ID NO: 68 - DNA, 18 bp, Artificial Sequence
  misc_feature: Synthetic Oligonucleotide
tctcccagcgcgcgccat

SEQ ID NO: 69 - DNA, 13 bp, Artificial Sequence
  misc_feature: Synthetic Oligonucleotide
tgtcgttgtcgtt

SEQ ID NO: 70 - DNA, 19 bp, Artificial Sequence
  misc_feature: Synthetic Oligonucleotide
tgtcgttgtcgttgtcgtt

SEQ ID NO: 71 - DNA, 25 bp, Artificial Sequence
  misc_feature: Synthetic Oligonucleotide
tgtcgttgtcgttgtcgttgtcgtt

SEQ ID NO: 72 - DNA, 21 bp, Artificial Sequence
  misc_feature: Synthetic Oligonucleotide
tgtcgtttgtcgtttgtcgtt

SEQ ID NO: 73 - DNA, 10 bp, Artificial Sequence
  misc_feature: Synthetic Oligonucleotide
nnnncgnnnn

SEQ ID NO: 74 - DNA, 8 bp, Artificial Sequence
  misc_feature: Synthetic Oligonucleotide
nnncgnnn

(19) United States
(12) Patent Application Publication
(10) Pub. No.: US 2005/0037985 A1
(43) Pub. Date: Feb. 17, 2005

(54) Methods and products for treating HIV infection

(76) Inventors: Arthur M. Krieg, Wellesley, MA (US); Dennis Klinman, Potomac, MD (US); Alfred D. Steinberg, Potomac, MD (US)

Correspondence Address: WOLF GREENFIELD & SACKS, PC;FEDERAL RESERVE PLAZA, 600 ATLANTIC AVENUE, BOSTON, MA, 02210-2211, US

(21) Appl. No.: 10/649,584

(22) Filed: Aug. 25, 2003

Related U.S. Application Data
(63) Continuation of application No. 09/931,583, filed on Aug. 16, 2001, now abandoned.
Application No. 09/931,583 is a continuation-in-part of application No. 09/415,142, filed on Oct. 9, 1999, now abandoned.
Application No. 09/415,142 is a division of application No. 08/386,063, filed on Feb. 7, 1995, now Pat. No. 6,194,388.
Application No. 08/386,063 is a continuation-in-part of application No. 08/276,358, filed on Jul. 15, 1994, now abandoned.

(51) Int. Cl. A61K 48/00

(52) U.S. Cl. 514/44

(57) ABSTRACT

Oligonucleotides containing unmethylated CpG dinucleotides and therapeutic utilities based on their ability to stimulate an immune response in a subject are disclosed. In particular, methods for treating HIV infection are disclosed.

### RELATED APPLICATIONS

This application claims priority to and is a continuation of co-pending U.S. Ser. No. 09/931,583 filed on Aug. 16, 2001, which is a continuation in part of co-pending U.S. Ser. No. 09/415,142 filed on Oct. 9, 1999 which claims priority to and is a divisional of U.S. Pat. No. 6,194,388 B1 which claims priority to and is a continuation-in-part of U.S. patent application Ser. No. 08/276,358, filed Jul. 15, 1994 which is now abandoned, the entire contents of which are hereby incorporated by reference.

### GOVERNMENT SUPPORT

The work resulting in this invention was supported in part by National Institute of Health Grant No. R29-AR42556-01. The U.S. Government may therefore be entitled to certain rights in the invention.

### FIELD OF THE INVENTION

The invention relates to oligonucleotides containing unmethylated CpG dinucleotides and therapeutic utilities based on their ability to stimulate an immune response in a subject. In particular, the invention relates to methods and products for treating HIV infection.

### BACKGROUND OF THE INVENTION

DNA Binds To Cell Membrane And Is Internalized: In the 1970's, several investigators reported the binding of high molecular weight DNA to cell membranes (Lerner, R. A., W. Meinke, and D. A. Goldstein. 1971. “Membrane-associated DNA in the cytoplasm of diploid human lymphocytes” PNAS USA 68:1212; Agrawal, S. K., R. W. Wagner, P. K. McAllister, and B. Rosenberg 1975 “Cell-surface-associated nucleic acid in tumorigenic cells made visible with platinum-pyrimidine complexes by electron microscopy” PNAS USA 72:928). In 1985 Bennett et al. presented the first evidence that DNA binding to lymphocytes is similar to a ligand receptor interaction: binding is saturable, competitive, and leads to DNA endocytosis and degradation (Bennett, R. M., G. T. Gabor, and M. M. Merritt 1985 “DNA binding to human leukocytes. Evidence for a receptor-mediated association, internalization, and degradation of DNA”. J. Clin. Invest. 76:2182). Like DNA, oligodeoxyribonucleotides (ODNs) are able to enter cells in a saturable, sequence independent, and temperature and energy dependent fashion (reviewed in Jaroszewski, J. W., and J. S. Cohen. 1991. “Cellular uptake of antisense oligodeoxynucleotides”. Advanced Drug Delivery Reviews 6:235; Akhtar, S., Y. Shoji, and R. L. Juliano. 1992. “Pharmaceutical aspects of the biological stability and membrane transport characteristics of antisense oligonucleotides”. In: Gene Regulation: Biology of Antisense RNA and DNA. R. P. Erickson, and J. G. Izant, eds. Raven Press, Ltd. N.Y., pp. 133; and Zhao, Q., T. Waldschmidt, E. Fisher, C. J. Herrera, and A. M. Krieg., 1994. “Stage specific oligonucleotide uptake in murine bone marrow B cell precursors”. Blood, 84:3660). No receptor for DNA or ODN uptake has yet been cloned, and it is not yet clear whether ODN binding and cell uptake occurs through the same or a different mechanism from that of high molecular weight DNA.

Lymphocyte ODN uptake has been shown to be regulated by cell activation. Spleen cells stimulated with the B cell mitogen LPS had dramatically enhanced ODN uptake in the B cell population, while spleen cells treated with the T cell mitogen Con A showed enhanced ODN uptake by T but not B cells (Krieg, A. M., F. Gmelig-Meyling, M. F. Gourley, W. J. Kisch, L. A. Chrisey, and A. D. Steinberg. 1991. “Uptake of oligodeoxyribonucleotides by lymphoid cells is heterogeneous and inducible”. Antisense Research and Development 1:161).

Immune Effects Of Nucleic Acids: Several polynucleotides have been extensively evaluated as biological response modifiers. Perhaps the best example is poly (I,C) which is a potent inducer of IFN production as well as a macrophage activator and inducer of NK activity (Talmadge, J. E., J. Adams, H. Phillips, M. Collins, B. Lenz, M. Schneider, E. Schlick, R. Ruffmann, R. H. Wiltrout, and M. A. Chirigos. 1985. “Immunomodulatory effects in mice of polyinosinic-polycytidylic acid complexed with poly-L:-lysine and carboxymethylcellulose”. Cancer Res. 45:1058; Wiltrout, R. H., R. R. Salup, T. A. Twilley, and J. E. Talmadge. 1985. “Immunomodulation of natural killer activity by polyribonucleotides”. J. Biol. Resp. Mod. 4:512; Krown, S. E. 1986. “Interferons and interferon inducers in cancer treatment”. Sem. Oncol. 13:207; and Ewel, C. H., S. J. Urba, W. C. Kopp, J. W. Smith II, R. G. Steis, J. L. Rossio, D. L. Longo, M. J. Jones, W. G. Alvord, C. M. Pinsky, J. M. Beveridge, K. L. McNitt, and S. P. Creekmore. 1992. “Polyinosinic-polycytidylic acid complexed with poly-L-lysine and carboxymethylcellulose in combination with interleukin 2 in patients with cancer: clinical and immnunological effects”. Canc. Res. 52:3005). It appears that this murine NK activation may be due solely to induction of IFN β secretion (Ishikawa, R., and C. A. Biron. 1993. “IFN induction and associated changes in splenic leukocyte distribution”. J. Immunol. 150:3713). This activation was specific for the ribose sugar since deoxyribose was ineffective. Its potent in vitro antitumor activity led to several clinical trials using poly (I,C) complexed with poly-L-lysine and carboxymethylcellulose (to reduce degradation by RNAse) (Talmadge, J. E., et al., 1985. cited supra; Wiltrout, R. H., et al., 1985. cited supra); Krown, S. E., 1986. cited supra); and Ewel, C. H., et al., 1992. cited supra). Unfortunately, toxic side effects have thus far prevented poly (I,C) from becoming a useful therapeutic agent.

Guanine ribonucleotides substituted at the C8 position with either a bromine or a thiol group are B cell mitogens and may replace “B cell differentiation factors” (Feldbush, T. L., and Z. K. Ballas. 1985. “Lymphokine-like activity of 8-mercaptoguanosine: induction of T and B cell differentiation”. J. Immunol. 134:3204; and Goodman, M. G. 1986. “Mechanism of synergy between T cell signals and C8-substituted guanine nucleosides in humoral immunity: B lymphotropic cytokines induce responsiveness to 8-mercaptoguanosine”. J. Immunol. 136:3335). 8-mercaptoguanosine and 8-bromoguanosine also can substitute for the cytokine requirement for the generation of MHC restricted CTL (Feldbush, T. L., 1985. cited supra), augment murine NK activity (Koo, G. C., M. E. Jewell, C. L. Manyak, N. H. Sigal, and L. S. Wicker. 1988. “Activation of murine natural killer cells and macrophages by 8-bromoguanosine”. J. Immunol. 140:3249), and synergize with IL-2 in inducing murine LAK generation (Thompson, R. A., and Z. K. Ballas. 1990. “Lymphokine-activated killer (LAK) cells. V. 8-Mercaptoguanosine as an IL-2-sparing agent in LAK generation”. J. Immunol. 145:3524). The NK and LAK augmenting activities of these C8-substituted guanosines appear to be due to their induction of IFN (Thompson, R. A., et al. 1990. cited supra). Recently, a 5′triphosphorylated thymidine produced by a mycobacterium was found to be mitogenic for a subset of human γδ T cells (Constant, P., F. Davodeau, M.-A. Peyrat, Y. Poquet, G. Puzo, M. Bonneville, and J.-J. Fournie. 1994. “Stimulation of human γδ T cells by nonpeptidic mycobacterial ligands” Science 264:267). This report indicated the possibility that the immune system may have evolved ways to preferentially respond to microbial nucleic acids.

Several observations suggest that certain DNA structures may also have the potential to activate lymphocytes. For example, Bell et al. reported that nucleosomal protein-DNA complexes (but not naked DNA) in spleen cell supernatants caused B cell proliferation and immunoglobulin secretion (Bell, D. A., B. Morrison, and P. VandenBygaart. 1990. “Immunogenic DNA-related factors”. J. Clin. Invest. 85:1487). In other cases, naked DNA has been reported to have immune effects. For example, Messina et al. have recently reported that 260 to 800 bp fragments of poly (dG)•(dC) and poly (dG•dC) were mitogenic for B cells (Messina, J. P., G. S. Gilkeson, and D. S. Pisetsky. 1993. “The influence of DNA structure on the in vitro stimulation of murine lymphocytes by natural and synthetic polynucleotide antigens”. Cell. Immunol. 147:148). Tokunaga, et al. have reported that dG•dC induces γ-IFN and NK activity (Tokunaga, S. Yamamoto, and K. Namba. 1988. “A synthetic single-stranded DNA, poly(dG,dC), induces interferon-(α/β and -γ, augments natural killer activity, and suppresses tumor growth” Jpn. J. Cancer Res. 79:682). Aside from such artificial homopolymer sequences, Pisetsky et al. reported that pure mammalian DNA has no detectable immune effects, but that DNA from certain bacteria induces B cell activation and immunoglobulin secretion (Messina, J. P., G. S. Gilkeson, and D. S. Pisetsky. 1991. “Stimulation of in vitro murine lymphocyte proliferation by bacterial DNA”. J. Immunol. 147:1759). Assuming that these data did not result from some unusual contaminant, these studies suggested that a particular structure or other characteristic of bacterial DNA renders it capable of triggering B cell activation. Investigations of mycobacterial DNA sequences have demonstrated that ODN which contain certain palindrome sequences can activate NK cells (Yamamoto, S., T. Yamamoto, T. Kataoka, E. Kuramoto, O. Yano, and T. Tokunaga. 1992. “Unique palindromic sequences in synthetic oligonucleotides are required to induce INF and augment INF-mediated natural killer activity”. J. Immunol. 148:4072; Kuramoto, E., O. Yano, Y. Kimura, M. Baba, T. Makino, S. Yamamoto, T. Yamamoto, T. Kataoka, and T. Tokunaga. 1992. “Oligonucleotide sequences required for natural killer cell activation”. Jpn. J. Cancer Res. 83:1128).

Several phosphorothioate modified ODN have been reported to induce in vitro or in vivo B cell stimulation (Tanaka, T., C. C. Chu, and W. E. Paul. 1992. “An antisense oligonucleotide complementary to a sequence in Iγ2b increases γ2b germline transcripts, stimulates B cell DNA synthesis, and inhibits immunoglobulin secretion”. J. Exp. Med. 175:597; Branda, R. F., A. L. Moore, L. Mathews, J. J. McCormack, and G. Zon. 1993. “Immune stimulation by an antisense oligomer complementary to the rev gene of HIV-1”. Biochem. Pharmacol. 45:2037; McIntyre, K. W., K. Lombard-Gillooly, J. R. Perez, C. Kunsch, U. M. Sarmiento, J. D. Larigan, K. T. Landreth, and R. Narayanan. 1993. “A sense phosphorothioate oligonucleotide directed to the initiation codon of transcription factor NFκβ T65 causes sequence-specific immune stimulation”. Antisense Res. Develop. 3:309; and Pisetsky, D. S., and C. F. Reich. 1993. “Stimulation of murine lymphocyte proliferation by a phosphorothioate oligonucleotide with antisense activity for herpes simplex virus”. Life Sciences 54: 101). These reports do not suggest a common structural motif or sequence element in these ODN that might explain their effects.

The CREB/ATF Family Of Transcription Factors And Their Role In Replication: The cAMP response element binding protein (CREB) and activating transcription factor (ATF) or CREB/ATF family of transcription factors is a ubiquitously expressed class of transcription factors of which 11 members have so far been cloned (reviewed in de Groot, R. P., and P. Sassone-Corsi: “Hormonal control of gene expression: Multiplicity and versatility of cyclic adenosine 3′,5′-monophosphate-responsive nuclear regulators”. Mol. Endocrin. 7:145, 1993; Lee, K. A. W., and N. Masson: “Transcriptional regulation by CREB and its relatives”. Biochim. Biophys. Acta 1174:221, 1993.). They all belong to the basic region/leucine zipper (bZip) class of proteins. All cells appear to express one or more CREB/ATF proteins, but the members expressed and the regulation of mRNA splicing appear to be tissue-specific. Differential splicing of activation domains can determine whether a particular CREB/ATF protein will be a transcriptional inhibitor or activator. Many CREB/ATF proteins activate viral transcription, but some splicing variants which lack the activation domain are inhibitory. CREB/ATF proteins can bind DNA as homo- or hetero-dimers through the cAMP response element, the CRE, the consensus form of which is the unmethylated sequence TGACGTC (binding is abolished if the CpG is methylated) (Iguchi-Ariga, S. M. M., and W. Schaffner: “CpG methylation of the cAMP-responsive enhancer/promoter sequence TGACGTCA abolishes specific factor binding as well as transcriptional activation”. Genes & Develop. 3:612, 1989.).

The transcriptional activity of the CRE is increased during B cell activation (Xie, H. T. C. Chiles, and T. L. Rothstein: “Induction of CREB activity via the surface Ig receptor of B cells”. J. Immunol. 151:880, 1993.). CREB/ATF proteins appear to regulate the expression of multiple genes through the CRE including immunologically important genes such as fos, jun B, Rb-1, IL-6, IL-1 (Tsukada, J., K. Saito, W. R. Waterman, A. C. Webb, and P. E. Auron: “Transcription factors NF-IL6 and CREB recognize a common essential site in the human prointerleukin 1 .beta. gene”. Mol. Cell. Biol. 14:7285, 1994; Gray, G. D., O. M. Hernandez, D. Hebel, M. Root, J. M. Pow-Sang, and E. Wickstrom: “Antisense DNA inhibition of tumor growth induced by c-Ha-ras oncogene in nude mice”. Cancer Res. 53:577, 1993), IFN-.beta. (Du, W., and T. Maniatis: “An ATF/CREB binding site protein is required for virus induction of the human interferon B gene”. Proc. Natl. Acad. Sci. USA 89:2150, 1992), TGF-β1 (Asiedu, C. K., L. Scott, R. K. Assoian, M. Ehrlich: “Binding of AP-1/CREB proteins and of MDBP to contiguous sites downstream of the human TGF-β1 gene”. Biochim. Biophys. Acta 1219:55, 1994.), TGF-β 2, class II MHC (Cox, P. M., and C. R. Goding: “An ATF/CREB binding motif is required for aberrant constitutive expression of the MHC class II DRa promoter and activation by SV40 T-antigen”. Nucl. Acids Res. 20:4881, 1992.), E-selectin, GM-CSF, CD-8α, the germline Igα constant region gene, the TCR V β gene, and the proliferating cell nuclear antigen (Huang, D., P. M. Shipman-Appasamy, D. J. Orten, S. H. Hinrichs, and M. B. Prystowsky: “Promoter activity of the proliferating-cell nuclear antigen gene is associated with inducible CRE-binding proteins in interleukin 2-stimulated T lymphocytes”. Mol. Cell. Biol. 14:4233, 1994.). In addition to activation through the cAMP pathway, CREB can also mediate transcriptional responses to changes in intracellular Ca⁺⁺ concentration (Sheng, M., G. McFadden, and M. E. Greenberg: “Membrane depolarization and calcium induce c-fos transcription via phosphorylation of transcription factor CREB”. Neuron 4:571, 1990).

The role of protein-protein interactions in transcriptional activation by CREB/ATF proteins appears to be extremely important. Activation of CREB through the cyclic AMP pathway requires protein kinase A (PKA), which phosphorylates CREB³⁴¹ on ser¹³³ and allows it to bind to a recently cloned protein, CBP (Kwok, R. P. S., J. R. Lundblad, J. C. Chrivia, J. P. Richards, H. P. Bachinger, R. G. Brennan, S. G. E. Roberts, M. R. Green, and R. H. Goodman: “Nuclear protein CBP is a coactivator for the transcription factor CREB”. Nature 370:223, 1994; Arias, J., A. S. Alberts, P. Brindle, F. X. Claret, T. Smea, M. Karin, J. Feramisco, and M. Montminy: “Activation of cAMP and mitogen responsive genes relies on a common nuclear factor”. Nature 370:226, 1994.). CBP in turn interacts with the basal transcription factor TFIIB causing increased transcription. CREB also has been reported to interact with dTAFII 110, a TATA binding protein-associated factor whose binding may regulate transcription (Ferreri, K., G. Gill, and M. Montminy: “The cAMP-regulated transcription factor CREB interacts with a component of the TFIID complex”. Proc. Natl. Acad. Sci. USA 91:1210, 1994.). In addition to these interactions, CREB/ATF proteins can specifically bind multiple other nuclear factors (Hoeffler, J. P., J. W. Lustbader, and C.-Y. Chen: “Identification of multiple nuclear factors that interact with cyclic adenosine 3′,5′-monophosphate response element-binding protein and activating transcription factor-2 by protein-protein interactions”. Mol. Endocrinol. 5:256, 1991) but the biologic significance of most of these interactions is unknown. CREB is normally thought to bind DNA either as a homodimer or as a heterodimer with several other proteins. Surprisingly, CREB monomers constitutively activate transcription (Krajewski, W., and K. A. W. Lee: “A monomeric derivative of the cellular transcription factor CREB functions as a constitutive activator”. Mol. Cell. Biol. 14:7204,1994.).

Aside from their critical role in regulating cellular transcription, it has recently been shown that CREB/ATF proteins are subverted by some infectious viruses and retroviruses, which require them for viral replication. For example, the cytomegalovirus immediate early promoter, one of the strongest known mammalian promoters, contains eleven copies of the CRE which are essential for promoter function (Chang, Y.-N., S. Crawford, J. Stall, D. R. Rawlins, K.-T. Jeang, and G. S. Hayward: “The palindromic series I repeats in the simian cytomegalovirus major immediate-early promoter behave as both strong basal enhancers and cyclic AMP response elements”. J. Virol. 64:264, 1990). At least some of the transcriptional activating effects of the adenovirus E1a protein, which induces many promoters, are due to its binding to the DNA binding domain of the CREB/ATF protein, ATF-2, which mediates E1a inducible transcription activation (Liu, F., and M. R. Green: “Promoter targeting by adenovirus E1a through interaction with different cellular DNA-binding domains”. Nature 368:520, 1994). It has also been suggested that E1a binds to the CREB-binding protein, CBP (Arany, Z., W. R. Sellers, D. M. Livingston, and R. Eckner: “E1a-associated p300 and CREB-associated CBP belong to a conserved family of coactivators”. Cell 77:799, 1994). Human T lymphotropic virus-I (HTLV-1), the retrovirus which causes human T cell leukemia and tropical spastic paresis, also requires CREB/ATF proteins for replication. In this case, the retrovirus produces a protein, Tax, which binds to CREB/ATF proteins and redirects them from their normal cellular binding sites to different DNA sequences (flanked by G- and C-rich sequences) present within the HTLV transcriptional enhancer (Paca-Uccaralertkun, S., L.-J. Zhao, N. Adya, J. V. Cross, B. R. Cullen, I. M. Boros, and C.-Z. Giam: “In vitro selection of DNA elements highly responsive to the human T-cell lymphotropic virus type I transcriptional activator, Tax”. Mol. Cell. Biol. 14:456, 1994; Adya, N., L.-J. Zhao, W. Huang, I. Boros, and C.-Z. Giam: “Expansion of CREB's DNA recognition specificity by Tax results from interaction with Ala-Ala-Arg at positions 282-284 near the conserved DNA-binding domain of CREB”. Proc. Natl. Acad. Sci. USA 91:5642,1994).

### SUMMARY OF THE INVENTION

The instant invention is based in part on the finding that certain oligonucleotides containing unmethylated cytosine-guanine (CpG) dinucleotides activate lymphocytes and other immune cells as evidenced by in vitro and in vivo data. Based on this finding, the invention features, in one aspect, methods for treating immune related disorders such as infectious disease using immunostimulatory oligonucleotides.

Thus, in one aspect the invention is a method for treating a subject by administering a CpG nucleic acid to a subject infected with human immunodeficiency virus (HIV) in an effective amount to treat HIV infection. In some embodiments the method further involves the step of administering an anti-HIV therapy.

According to another aspect of the invention a method for treating a subject by administering a CpG nucleic acid and an anti-HIV therapy to a subject infected with human immunodeficiency virus (HIV) in an effective amount to treat HIV infection is provided.

In one embodiment the anti-HIV therapy is an inhibitor of HIV replication, such as a protease inhibitor, e.g., HAART. In another embodiment the anti-HIV therapy is a cytokine or a chemokine. The cytokine may optionally be a T-cell activating cytokine, such as IL-2. The chemokine may be RANTES or MIP-1α.

In some embodiments the anti-HIV therapy or CpG nucleic acid are administered in a sub-therapeutic dosage and wherein the combination of the sub-therapeutic dose of the anti-HIV therapy and the CpG nucleic acid produce a therapeutic result in the treatment of HIV infection.

The anti-HIV therapy may be administered at the same time as the CpG nucleic acid. Alternatively, the anti-HIV therapy may be administered prior to the CpG nucleic acid. In some embodiments the anti-HIV therapy is administered prior to the initial administration of CpG nucleic acid and the anti-HIV therapy is continued during the administration of the CpG nucleic acid. Optionally the anti-HIV therapy is terminated, e.g., at least one week after the initial administration of CpG. In other embodiments the CpG nucleic acid is administered prior to the initial administration of anti-HIV therapy and the CpG nucleic acid is continued during the administration of the anti-HIV therapy.

According to other embodiments the CpG nucleic acid and the anti-HIV therapy may be administered in alternating cycles e.g., monthly cycles.

The subject, in some embodiments, may be treated with an anti-HIV therapy and a nucleic acid which is optionally an IFN-α-inducing CpG nucleic acid. Optionally the subject may be administered a vaccine and a CpG nucleic acid as an adjuvant before, at the same time as, or after the anti-HIV therapy and the CpG nucleic acid. The CpG nucleic acid that is administered with the vaccine may be the same as or different than the CpG nucleic acid that is administered with the anti-HIV therapy or some combination thereof. In either case, the CpG nucleic acid may be an adjuvant-type CpG nucleic acid, an IFN-α-inducing CpG nucleic acid, a combination thereof or another type of CpG nucleic acid.

At some time during the therapy the anti-HIV therapy may be stopped. When the anti-HIV therapy is stopped the CpG nucleic acid therapy and/or the vaccine may still be administered to the subject or may be stopped. Any of these three therapies, the anti-HIV therapy, the CpG nucleic acid and/or the vaccine may be stopped and re-started at different intervals or at the same time. The stopping and starting of the therapy might be performed at routine intervals or optionally may be performed in response to clinical progress of a particular subject.

The method in some embodiments involves the step of administering a non-steroidal anti-inflammatory agent to the subject. Non-steroidal anti-inflammatory agent include but are not limited to Piroxicam, Mefenamic acid, Nabumetone, Sulindac, Tolmetin, Ketorolac, Rofecoxib, Diclofenac, Naproxen, Flurbiprofen, Celecoxib, Oxaprozin, Diflunisal, Etodolac, Fenoprofen, Ibuprofen, Indomethacin, Ketoprofen, Etodolac, and Meloxicam.

In another aspect the invention is a method for treating a subject by administering a vaccine and a CpG nucleic acid as an adjuvant to a subject infected with or at risk of being infected with human immunodeficiency virus (HIV) in an effective amount to treat or prevent HIV infection.

The CpG nucleic acid may be administered at the same time as the vaccine in some embodiments. In other embodiments the CpG nucleic acid is administered before the vaccine.

In the methods of the invention the CpG nucleic acid may be any type of nucleic acid containing a CpG motif. It may or may not include a palindrome. In some embodiments the CpG nucleic acid is an adjuvant-type CpG nucleic acid. The adjuvant-type CpG nucleic acid may have a sequence including at least the following formula: 5′[TCN₁TN₂X₁X₂CGTT]N₃[X₁X₂CGTT]N₄[X₁X₂CGTT] 3′ (SEQ ID NO: 33), wherein N₄ is about 0-26 bases with the proviso that N₄ does not contain a CCGG quadmer or more than one CCG or CGG trimer. In some embodiments N₄ is selected from the group consisting of nothing, any nucleotide, C, T, TT, TTT, TTTT, and TC. In other embodiments N₃ and N₄ are both TT, X₂ is T, and/or X₁ is G.

In some embodiments the adjuvant-type CpG nucleic acid has a sequence including one of at least the following formulas:

| | |
|---|---|
| GTCGTTN₃GTCGTTN₄GTCGTT. | (SEQ ID NO:34) |
| TCGTCGTTTTGTCGTTTTGTCGTT. | (SEQ ID NO:29) |
| TCGTCGTTTTGTCGTTTTGTCGTTTTT. | (SEQ ID NO:35) |
| TCGTCGTTTTGTCGTTTTGTCGTTCCC. | (SEQ ID NO:36) |
| TCGTCGTTTTGTCGTTTTGTCGTTAAA. | (SEQ ID NO:37) |
| TCGTCGTTTTGTCGTTTTGTCGTT. | (SEQ ID NO:38) |

The CpG nucleic acid alternatively may be an IFN-α-inducing CpG nucleic acid. In some embodiments the IFN-α-inducing CpG nucleic acid comprises the following sequence

| | |
|---|---|
| 5′ Y₁N₁X₁X₂CGX₃X₄N₂Y₂ 3′, | (SEQ ID NO:73) |

wherein G is guanine; C is unmethylated cytosine; X₁, X₂, X₃, and X₄ independently are single nucleotides; N₁ and N₂ are independently nucleic acid molecules each having between 0 and 20 nucleotides; N₁X₁X₂CGX₃X₄N₂ (SEQ ID NO: 74) includes a palindrome at least 6 nucleotides long that contains at least one CG; Y₁ is a nucleic acid molecule having between 1 and 8 nucleotides comprising at least one modified internucleotide linkage; and Y₂ is independently a nucleic acid molecule having between 3 and 8 nucleotides comprising at least 3 consecutive Gs and at least one modified internucleotide linkage. Optionally, at least one modified internucleotide linkage is a phosphorothioate modified linkage. In some embodiments Y₁ includes at least 3 Gs or is all Gs and/or Y₂ includes at least 4 Gs or is all Gs. In yet other embodiments Y₁ includes between two and five modified internucleotide linkages and Y₂ includes between two and five modified internucleotide linkages. Preferably the palindrome has a phosphodiester backbone.

Thus, in some embodiments the CpG nucleic acid may be an adjuvant type or an IFNα inducing CpG nucleic acid or a combination of both. Preferably the CpG nucleic acid has less than or equal to 100 nucleotides. The type of CpG nucleic acid used with a vaccine is not limited to an adjuvant type CpG nucleic acid. In some cases it may be preferable to use a non-adjuvant CpG nucleic acid or a combination of an adjuvant and a non-adjuvant CpG nucleic acid. In some embodiments an IFN-α-inducing CpG nucleic acid is administered with the vaccine. This may result in the production of a higher cytotoxic T lymphocyte (CTL) response.

Each of the limitations of the invention can encompass various embodiments of the invention. It is, therefore, anticipated that each of the limitations of the invention involving any one element or combinations of elements can be included in each aspect of the invention.

### DETAILED DESCRIPTION

As used herein, the following terms and phrases shall have the meanings set forth below:

The terms “nucleic acid” and “oligonucleotide” are used interchangeably to mean multiple nucleotides (i.e. molecules comprising a sugar (e.g. ribose or deoxyribose) linked to a phosphate group and to an exchangeable organic base, which is either a substituted pyrimidine (e.g. cytosine (C), thymine (T) or uracil (U)) or a substituted purine (e.g. adenine (A) or guanine (G)). The term “oligonucleotide” as used herein refers to both oligoribonucleotides (ORNs) and oligodeoxyribonucleotides (ODNs). The term “oligonucleotide” shall also include oligonucleosides (i.e. an oligonucleotide minus the phosphate) and any other organic base containing polymer. Oligonucleotides can be obtained from existing nucleic acid sources (e.g., genomic or cDNA, referred to as isolated nucleic acids), but are preferably synthetic (e.g. produced by oligonucleotide synthesis).

An “immunostimulatory oligonucleotide”, “immunostimulatory CpG containing oligonucleotide”, “CpG ODN” or a “CpG nucleic acid” refer to a nucleic acid which includes at least one unmethylated CpG dinucleotide. A nucleic acid containing at least one unmethylated CpG dinucleotide is a nucleic acid molecule which contains an unmethylated cytosine in a cytosine-guanine dinucleotide sequence (i.e. “CpG DNA” or DNA containing a 5′ cytosine followed by 3′ guanosine and linked by a phosphate bond) and activates the immune system. The CpG nucleic acids can be double-stranded or single-stranded. Generally, double-stranded molecules are more stable in vivo, while single-stranded molecules have increased immune activity. Thus in some aspects of the invention it is preferred that the nucleic acid be single stranded and in other aspects it is preferred that the nucleic acid be double stranded. The terms CpG nucleic acid or CpG oligonucleotide as used herein refer to an immunostimulatory CpG nucleic acid unless otherwise indicated. The entire immunostimulatory nucleic acid can be unmethylated or portions may be unmethylated but at least the C of the 5′ CG 3′ must be unmethylated.

In one preferred embodiment the invention provides an immunostimulatory nucleic acid which is a CpG nucleic acid represented by at least the formula:
5′X₁X₂CGX₃X₄3′
wherein X₁, X₂, X₃, and X₄ are nucleotides. In one embodiment X₂ is adenine, guanine, cytosine, or thymine. In another embodiment X₃ is cytosine, guanine, adenine, or thymine. In other embodiments X₂ is adenine, guanine, or thymine and X₃ is cytosine, adenine, or thymine.

An “adjuvant-type CpG nucleic acid” as used herein is a CpG nucleic acid having at least the formula:

| | |
|---|---|
| 5′ TCN₁TN₂X₁X₂CGX₃X₄ 3′. | (SEQ ID NO:27) |

In some embodiments, the adjuvant-type CpG nucleic acid has a base sequence including at least the following formula:

| | |
|---|---|
| 5′ TCN₁TX₁X₂CGX₃X₄ 3′. | (SEQ ID NO:28) |

In the forgoing embodiments of the adjuvant-type CpG nucleic acid, N₁, and N₂ are about 0-25 nucleotides, G is guanine, C is unmethylated cytosine, and X₁, X₂, X₃, and X₄ independently are single nucleotides. In one embodiment X₁X₂ are nucleotides selected from the group consisting of: TpT, TpG, TpA, GpT, GpG, GpA, ApT, ApG, ApA, CpT, and CpA; and X₃X₄ are nucleotides selected from the group consisting of: TpT, TpG, TpA, TpC, ApT, ApG, ApA, ApC, CpT, CpA, and CpC.

In some embodiments, wherein the adjuvant-type CpG nucleic acid has a backbone comprising at least one modified internucleotide linkage. In certain embodiments, the at least one modified internucleotide linkage is a phosphorothioate modified linkage and in other embodiments the adjuvant-type CpG nucleic acid has a backbone made up entirely of modified internucleotide linkages. In some embodiments of the invention, the adjuvant-type CpG nucleic acid is (TCGTCGTTTTGTCGTTTTGTCGTT) (SEQ ID NO: 29).

X₁X₂ in another embodiment are nucleotides selected from the group consisting of: GpT, GpG, GpA and ApA and X₃X₄ are nucleotides selected from the group consisting of: TpT, CpT or GpT. In yet other embodiments, X₁X₂ are GpA and X₃X₄ are TpT. In some embodiments, X₁X₂ are both purines and X₃X₄ are both pyrimidines. In other embodiments, X₁X₂ are GpA and X₃X₄ are both pyrimidines. In certain embodiments, the nucleic acid is 8 to 40 nucleotides in length, and in other embodiments is less than or equal to 100 nucleotides in length.

In another preferred embodiment, the adjuvant-type CpG nucleic acid is represented by at least the formula:

| | |
|---|---|
| 5′ [TCN₁TN₂X₁X₂CGTT] 3′, | (SEQ ID NO:31) |

wherein X₁X₂ is selected from the group consisting of GT, GA, and AT, wherein at least one nucleotide has a phosphate backbone modification, and wherein C of CpG is unmethylated.

In some embodiments, X₁ is G. In some embodiments, the adjuvant-type CpG nucleic acid has a sequence including at least the following formula:

| | |
|---|---|
| 5′ [TCN₁TN₂X₁X₂CGTT]N₃[X₁X₂CGTT] 3′, | (SEQ ID NO:32) |

wherein N₃ is about 0-26 nucleotides, N₃ may not in some embodiments contain a CCGG quadmer or more than one CCG or CGG trimer. N₃ may optionally be selected from the group consisting of nothing, any nucleotide, C, T, TT, TTT, TTTT, and TC. In some embodiments, N₃ is a single pyrimidine. In other embodiments, N₃ is at least two pyrimidines. In certain embodiments, N₃ is 0 nucleotides. In other embodiments N₃ is 1 nucleotide. In yet other embodiments, N₃ is at least 2 nucleotides.

In some embodiments, the adjuvant-type CpG nucleic acid has a sequence including at least the following formula:

| | |
|---|---|
| 5′ [TCN₁TN₂X₁X₂CGTT]N₃[X₁X₂CGTT]N₄[X₁X₂CGTT] 3′, | (SEQ ID NO:33) |

wherein N₄ is about 0-26 bases and with the proviso that N₄ may not contain a CCGG quadmer or more than one CCG or CGG trimer. In some embodiments, N₄ is selected from the group consisting of nothing, any nucleotide, C, T, TT, TTT, TTTT, and TC. In certain embodiments, N₃ and N₄ are both TT. In some embodiments, X₂ is T. In some embodiments, X₁ is G. In certain embodiments, the adjuvant-type CpG nucleic acid has a sequence including at least one of the following formulas:

| | |
|---|---|
| [GTCGTT]N₃[GTCGTT]N₄[GTCGTT] | (SEQ ID NO:34) |

Optionally, the immunostimulatory nucleic acid has a sequence including at least the following formula:

| | |
|---|---|
| TCGTCGTTTTGTCGTTTTGTCGTT, | (SEQ ID NO:29) |
| TCGTCGTTTTGTCGTTTTGTCGTTTTT, | (SEQ ID NO:35) |
| TCGTCGTTTTGTCGTTTTGTCGTTCCC, | (SEQ ID NO:36) |
| TCGTCGTTTTGTCGTTTTGTCGTTAAA, | (SEQ ID NO:37) or |
| TCGTCGTTTTGTCGTTTTGTCGTT. | (SEQ ID NO:38) |

Exemplary sequences include but are not limited to these immunostimulatory sequence shown in Table 1.

TABLE 1
Exemplary CpG nucleic acids

| | |
|---|---|
| ATCGACTCTCGAGCGTTCTC | (SEQ ID NO:39) |
| ATCGACTCTCGAGCGTTZTC | (SEQ ID NO:40) |
| TCCACGACGTTTTCGACGTT | (SEQ ID NO:41) |
| TCCATAACGTTCCTGATGCT | (SEQ ID NO:42) |
| TCCATAGCGTTCCTAGCGTT | (SEQ ID NO:43) |
| TCCATCACGTGCCTGATGCT | (SEQ ID NO:44) |
| TCCATGACGGTCCTGATGCT | (SEQ ID NO:45) |
| TCCATGACGTCCCTGATGCT | (SEQ ID NO:46) |
| TCCATGACGTTCCTGACGTT | (SEQ ID NO:47) |
| TCCATGACGTTCCTGATGCT | (SEQ ID NO:48) |
| TCCATGCCGGTCCTGATGCT | (SEQ ID NO:49) |
| TCCATGCGTTGCGTTGCGTT | (SEQ ID NO:50) |
| TCCATGGCGGTCCTGATGCT | (SEQ ID NO:51) |
| TCCATGTCGATCCTGATGCT | (SEQ ID NO:52) |
| TCCATGTCGCTCCTGATGCT | (SEQ ID NO:53) |
| TCCATGTCGGTCCTGATGCT | (SEQ ID NO:54) |
| TCCATGTCGGTCCTGCTGAT | (SEQ ID NO:55) |
| TCCATGTCGGTZCTGATGCT | (SEQ ID NO:56) |
| TCCATGTCGTTCCTGATGGT | (SEQ ID NO:57) |
| TCCATGTCGTTCCTGTCGTT | (SEQ ID NO:58) |
| TCCTGACGTTCCTGACGTT | (SEQ ID NO:59) |
| TCCTGTCGTTCCTGTCGTT | (SEQ ID NO:60) |
| TCCTGTCGTTCCTTGTCGTT | (SEQ ID NO:61) |
| TCCTGTCGTTTTTTGTCGTT | (SEQ ID NO:62) |
| TCCTTGTCGTTCCTGTCGTT | (SEQ ID NO:63) |
| TCGTCGCTGTTGTCGTTTCTT | (SEQ ID NO:64) |
| TCGTCGTCGTCGTT | (SEQ ID NO:65) |
| TCGTCGTTGTCGTTGTCGTT | (SEQ ID NO:66) |
| TCGTCGTTGTCGTTTTGTCGTT | (SEQ ID NO:67) |
| TCGTCGTTTTGTCGTTTTGTCGTT | (SEQ ID NO:29) |
| TCTCCCAGCGCGCGCCAT | (SEQ ID NO:68) |
| TGTCGTTGTCGT | (SEQ ID NO:69) |
| TGTCGTTGTCGTTTGTCGTT | (SEQ ID NO:70) |
| TGTCGTTGTCGTTGTCGTTGTCGTT | (SEQ ID NO:71) |
| TGTCGTTTGTCGTTTGTCGTT | (SEQ ID NO:72) |

In a preferred embodiment of the invention, the IFN-α-inducing CpG nucleic acid comprises a base sequence

| | |
|---|---|
| 5′ Y₁N₁X₁X₂CGX₃X₄N₂Y₂ 3′, | (SEQ ID NO: 73) |

wherein G is guanine; C is unmethylated cytosine; X₁, X₂, X₃, and X₄ independently are single nucleotides; N₁ and N₂ are independently nucleic acid molecules each having between 0 and 20 nucleotides; N₁X₁X₂CGX₃X₄N₂ (SEQ ID NO: 74) includes a palindrome at least 6 nucleotides long that contains at least one CG; Y₁ is a nucleic acid molecule having between 1 and 8 nucleotides comprising at least one modified internucleotide linkage; and Y₂ is independently a nucleic acid molecule having between 3 and 8 nucleotides comprising at least 3 consecutive Gs and at least one modified internucleotide linkage. In some embodiments, at least one modified internucleotide linkage is a phosphorothioate modified linkage. In certain embodiments, Y₁ is comprised of at least 3 Gs. In certain embodiments, Y₁ is comprised of at least 4 Gs. In other embodiments, Y₁ is comprised of at least 7 Gs. In some embodiments Y₁ is comprised of all Gs. In some embodiments Y₂ is comprised of at least 4 Gs. In other embodiments, Y₂ is comprised of at least 7 Gs. In yet other embodiments, Y₂ is comprised of all Gs. In some embodiments, Y₁ includes at least two modified internucleotide linkages and Y₂ includes at least two modified internucleotide linkages. In certain embodiments, Y₁ includes between two and five modified internucleotide linkages and Y₂ includes between two and five modified internucleotide linkages. In some embodiments, the palindrome has a phosphodiester backbone. In other embodiments, the IFN-α-inducing CpG nucleic acid as a whole has a backbone made up entirely of modified internucleotide linkages. In certain embodiments, the IFN-α-inducing CpG nucleic acid is ODN 2306 (ggGGACGTCGACGTggggG) (SEQ ID NO: 30).

For facilitating uptake into cells, the immunostimulatory nucleic acids are preferably in the range of 6 to 100 bases in length. However, nucleic acids of any size greater than 6 nucleotides (even many kb long) are capable of inducing an immune response according to the invention if sufficient immunostimulatory motifs are present. Preferably the immunostimulatory nucleic acid is in the range of between 8 and 100 and in some embodiments between 8 and 50 or 8 and 30 nucleotides in size.

“Palindromic sequence” shall mean an inverted repeat (i.e. a sequence such as ABCDEE′D′C′B′A′ in which A and A′ are bases capable of forming the usual Watson-Crick base pairs. In vivo, such sequences may form double stranded structures. In one embodiment the CpG nucleic acid contains a palindromic sequence. A palindromic sequence used in this context refers to a palindrome in which the CpG is part of the palindrome, and preferably is the center of the palindrome. In another embodiment the CpG nucleic acid is free of a palindrome. An immunostimulatory nucleic acid that is free of a palindrome is one in which the CpG dinucleotide is not part of a palindrome. Such an oligonucleotide may include a palindrome in which the CpG is not the center of the palindrome.

For use in the instant invention, the CpG nucleic acids can be synthesized de novo using any of a number of procedures well known in the art. Such compounds are referred to as “synthetic nucleic acids.” For example, the b-cyanoethyl phosphoramidite method (Beaucage, S. L., and Caruthers, M. H., Tet. Let. 22:1859, 1981); nucleoside H-phosphonate method (Garegg et al., Tet. Let. 27:4051-4054, 1986; Froehler et al., Nucl. Acid. Res. 14:5399-5407, 1986, ; Garegg et al., Tet. Let. 27:4055-4058, 1986, Gaffney et al., Tet. Let. 29:2619-2622, 1988). These chemistries can be performed by a variety of automated oligonucleotide synthesizers available in the market. These nucleic acids are referred to as synthetic nucleic acids. Alternatively, immunostimulatory nucleic acids can be produced on a large scale in plasmids, (see Sambrook, T., et al., “Molecular Cloning: A Laboratory Manual”, Cold Spring Harbor laboratory Press, N.Y., 1989) and separated into smaller pieces or administered whole. Nucleic acids can be prepared from existing nucleic acid sequences using known techniques, such as those employing restriction enzymes, exonucleases or endonucleases. Nucleic acids prepared in this manner are referred to as isolated nucleic acids. The term “CpG nucleic acid” encompasses both synthetic and isolated immunostimulatory nucleic acids.

For use in vivo, nucleic acids are preferably relatively resistant to degradation (e.g., are stabilized). A “stabilized nucleic acid molecule” shall mean a nucleic acid molecule that is relatively resistant to in vivo degradation (e.g. via an exo- or endo-nuclease). Stabilization can be a function of length or secondary structure. Immunostimulatory nucleic acids that are tens to hundreds of kbs long are relatively resistant to in vivo degradation. For shorter immunostimulatory nucleic acids, secondary structure can stabilize and increase their effect. For example, if the 3′ end of a nucleic acid has self-complementarity to an upstream region, so that it can fold back and form a sort of stem loop structure, then the nucleic acid becomes stabilized and therefore exhibits more biological in vivo activity.

Alternatively, nucleic acid stabilization can be accomplished via backbone modifications. Preferred stabilized nucleic acids of the instant invention have a modified backbone. It has been demonstrated that modification of the nucleic acid backbone provides enhanced activity of the immunostimulatory nucleic acids when administered in vivo. One type of modified backbone is a phosphate backbone modification. Immunostimulatory nucleic acids, including at least two phosphorothioate linkages at the 5′ end of the oligonucleotide and multiple phosphorothioate linkages at the 3′ end, preferably 5, can in some circumstances provide maximal activity and protect the nucleic acid from degradation by intracellular exo- and endo-nucleases. Other phosphate modified nucleic acids include phosphodiester modified nucleic acids, combinations of phosphodiester and phosphorothioate nucleic acids, methylphosphonate, methylphosphorothioate, phosphorodithioate, and combinations thereof. Although not intending to be bound by any particular theory, it is believed that these phosphate modified nucleic acids may show more stimulatory activity due to enhanced nuclease resistance, increased cellular uptake, increased protein binding, and/or altered intracellular localization.

Modified backbones such as phosphorothioates may be synthesized using automated techniques employing either phosphoramidate or H-phosphonate chemistries. Aryl-and alkyl-phosphonates can be made, e.g., as described in U.S. Pat. No. 4,469,863. Alkylphosphotriesters, in which the charged oxygen moiety is alkylated as described in U.S. Pat. No. 5,023,243 and European Patent No. 092,574, can be prepared by automated solid phase synthesis using commercially available reagents. Methods for making other DNA backbone modifications and substitutions have been described (Uhlmann, E. and Peyman, A., Chem. Rev. 90:544, 1990; Goodchild, J., Bioconjugate Chem. 1:165, 1990).

Another type of modified backbone, useful according to the invention, is a peptide nucleic acid. The backbone is composed of aminoethylglycine and supports bases which provide the DNA character. The backbone does not include any phosphate and thus may optionally have no net charge. The lack of charge allows for stronger DNA-DNA binding because the charge repulsion between the two strands does not exist. Additionally, because the backbone has an extra methylene group, the oligonucleotides are enzyme/protease resistant. Peptide nucleic acids can be purchased from various commercial sources, e.g., Perkin Elmer, or synthesized de novo.

Another class of backbone modifications include 2′-O-methylribonucleosides (2′-Ome). These types of substitutions are described extensively in the prior art and in particular with respect to their immunostimulating properties in Zhao et al., Bioorganic and Medicinal Chemistry Letters, 1999, 9:24:3453. Zhao et al. describes methods of preparing 2′-Ome modifications to nucleic acids.

The nucleic acid molecules of the invention may include naturally-occurring or synthetic purine or pyrimidine heterocyclic bases as well as modified backbones. Purine or pyrimidine heterocyclic bases include, but are not limited to, adenine, guanine, cytosine, thymidine, uracil, and inosine. Other representative heterocyclic bases are disclosed in U.S. Pat. No. 3,687,808, issued to Merigan, et al. The terms “purines” or “pyrimidines” or “bases” are used herein to refer to both naturally-occurring or synthetic purines, pyrimidines or bases.

Other stabilized nucleic acids include non-ionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated. Nucleic acids which contain diol, such as tetraethyleneglycol or hexaethyleneglycol, at either or both termini have also been shown to be substantially resistant to nuclease degradation.

The immunostimulatory nucleic acids having backbone modifications useful according to the invention in some embodiments are S- or R-chiral immunostimulatory nucleic acids. An “S chiral immunostimulatory nucleic acid” as used herein is an immunostimulatory nucleic acid wherein at least two nucleotides have a backbone modification forming a chiral center and wherein a plurality of the chiral centers have S chirality. An “R chiral immunostimulatory nucleic acid” as used herein is an immunostimulatory nucleic acid wherein at least two nucleotides have a backbone modification forming a chiral center and wherein a plurality of the chiral centers have R chirality. The backbone modification may be any type of modification that forms a chiral center. The modifications include but are not limited to phosphorothioate, methylphosphonate, methylphosphorothioate, phosphorodithioate, 2′-Ome and combinations thereof.

The S- and R-chiral immunostimulatory nucleic acids may be prepared by any method known in the art for producing chirally pure oligonucleotides. Stec et al teach methods for producing stereopure phosphorothioate oligodeoxynucleotides using an oxathiaphospholane. (Stec, W. J., et al., 1995, J. Am. Chem. Soc., 117:12019). Other methods for making chirally pure oligonucleotides have been described by companies such as ISIS Pharmaceuticals. U.S. Pat. Nos. which disclose methods for generating stereopure oligonucleotides include 5,883,237, 5,837,856, 5,599,797, 5,512,668, 5,856,465, 5,359,052, 5,506,212, 5,521,302 and 5,212,295, each of which is hereby incorporated by reference in its entirety.

An “oligonucleotide delivery complex” shall mean an oligonucleotide associated with (e.g. ionically or covalently bound to; or encapsulated within) a targeting means (e.g. a molecule that results in higher affinity binding to target cell (e.g. B-cell and natural killer (NK) cell) surfaces and/or increased cellular uptake by target cells). Examples of oligonucleotide delivery complexes include oligonucleotides associated with: a sterol (e.g. cholesterol), a lipid (e.g. a cationic lipid, virosome or liposome), or a target cell specific binding agent (e.g. a ligand recognized by target cell specific receptor). Preferred complexes must be sufficiently stable in vivo to prevent significant uncoupling prior to internalization by the target cell. However, the complex should be cleavable under appropriate conditions within the cell so that the oligonucleotide is released in a functional form.

An “immune system deficiency” shall mean a disease or disorder in which the subject's immune system is not functioning in normal capacity or in which it would be useful to boost a subject's immune response for example to eliminate a tumor or cancer (e.g. tumors of the brain, lung (e.g. small cell and non-small cell), ovary, breast, prostate, colon, as well as other carcinomas and sarcomas) or a viral (e.g. HIV, herpes), fungal (e.g. Candida sp.), bacterial or parasitic (e.g. Leishmania, Toxoplasma) infection in a subject.

A “disease associated with immune system activation” shall mean a disease or condition caused or exacerbated by activation of the subject's immune system. Examples include systemic lupus erythematosus, sepsis and autoimmune diseases such as rheumatoid arthritis and multiple sclerosis.

A “subject” shall mean a human or vertebrate animal including a dog, cat, horse, cow, pig, sheep, goat, chicken, monkey, rat, mouse. Preferably the subject is a human or other vertebrate that is capable of being infected with an immunodeficiency virus.

In particular, the compounds of the invention are useful for treating and preventing HIV infection in a subject. HIV infection may be treated using a CpG nucleic acid alone or in combination with another therapeutic such as an anti-HIV therapy. An anti-HIV therapy, as used herein is any therapeutic that is useful for reducing viral load, preventing viral infection, prolonging the asymptotic phase of HIV infection, or prolonging the life of a subject infected with HIV. Anti-HIV therapies include but are not limited to inhibitors of HIV replication, such as protease inhibitors, e.g., HAART; cytokines; and chemokines.

In some instances, a sub-therapeutic dosage of either the CpG nucleic acid or the anti-HIV therapy, or a sub-therapeutic dosage of both, is used in the treatment of a subject having or at risk of developing HIV. As an example, it has been discovered according to the invention, that when the two classes of drugs are used together, the anti-HIV therapy can be administered in a sub-therapeutic dose and still produce a desirable therapeutic result. A “sub-therapeutic dose” as used herein refers to a dosage which is less than that dosage which would produce a therapeutic result in the subject if administered in the absence of the other agent. Thus, the sub-therapeutic dose of an anti-HIV therapy is one which would not produce the desired therapeutic result in the subject in the absence of the administration of the CpG nucleic acid. Therapeutic doses of anti-HIV therapy are well known in the field of medicine for the treatment of HIV. These dosages have been extensively described in medical references relied upon by the medical profession as guidance for the treatment of HIV. Therapeutic dosages of CpG nucleic acids have also been described in the art and methods for identifying therapeutic dosages in subjects are described in more detail herein.

In other aspects, the method of the invention involves administering a dose of an anti-HIV therapy to a subject, without inducing side effects, due to the administration of a CpG nucleic acid. Ordinarily, when an anti-HIV therapy is administered to a subject in a therapeutic dose, a variety of side effects can occur. The severity of these side effects, in some instances, increase with increasing dosage of the anti-HIV therapy. It is for this reason that anti-HIV therapy is sometimes administered at the lowest possible therapeutic dose in order to prevent the occurrence of the adverse side effects. Additionally, some patients are non-compliant when high therapeutic doses are administered as a result of the side effects, no matter what therapeutic benefits are derived. However, it was discovered, according to the invention, that high doses of anti-HIV therapy which ordinarily induce side effects can be administered with reduced side effects as long as the subject also receives a CpG nucleic acid. The type and extent of the side effects ordinarily induced by the anti-HIV therapy will depend on the particular anti-HIV therapy used. Thus the invention provides methods for reducing side effects resulting from the administration of low or high therapeutic doses of anti-HIV therapy.

Some aspects of the invention call for the administration of a CpG nucleic acid in an effective amount to inhibit the induction of side effects by an anti-HIV therapy when the anti-HIV therapy is administered in a dose which ordinarily, if administered by itself, would induce side effects. An effective amount of an CpG nucleic acid to inhibit the induction of side effects may be defined as the effective amount to inhibit a hypersensitivity reaction. The effective amount to inhibit the induction of side effects may also be that amount which inhibits increases in bone density.

For any compound described herein a therapeutically effective amount can be initially determined from cell culture assays. In particular, the effective amount of CpG nucleic acid can be determined using in vitro stimulation assays. The stimulation index of the CpG nucleic acid can be compared to that of previously tested immunostimulatory acids. The stimulation index can be used to determine an effective amount of the particular oligonucleotide for the particular subject, and the dosage can be adjusted upwards or downwards to achieve the desired levels in the subject.

Therapeutically effective amounts can also be determined in animal studies. For instance, the effective amount of CpG nucleic acid and anti-HIV therapy to induce a therapeutic response can be assessed using in vivo assays of viral load. Relevant animal models include primates infected with simian immunodeficiency virus (SIV). Generally, a range of CpG nucleic acid doses are administered to the animal along with a range of anti-HIV therapy doses. Reduction in viral load in the animals following the administration of the active agnets is indicative of the ability to reduce the viral load and thus treat HIV infection.

A therapeutically effective dose can also be determined from human data for CpG nucleic acids which have been tested in humans (human clinical trials have been initiated) and for compounds which are known to exhibit similar pharmacological activities, such as other adjuvants for vaccination purposes.

The applied dose of both the CpG nucleic acid and the anti-HIV therapy can be adjusted based on the relative bioavailability and potency of the administered compounds. Adjusting the dose to achieve maximal efficacy based on the methods described above and other methods are well within the capabilities of the ordinarily skilled artisan. Most of the anti-HIV therapies have been identified. These amounts can be adjusted when they are combined with CpG nucleic acids by routine experimentation.

Subject doses of the compounds described herein typically range from about 0.1 μg to 10,000 mg, more typically from about 1 μg/day to 8000 mg, and most typically from about 10 μg to 100 μg. Stated in terms of subject body weight, typical dosages range from about 0.1 μg to 20 mg/kg/day, more typically from about 1 to 10 mg/kg/day, and most typically from about 1 to 5 mg/kg/day.

In other embodiments of the invention, the CpG nucleic acid and the anti-HIV therapy may be administered at the same time or in alternating cycles or any other therapeutically effective schedule. “Alternating cycles” as used herein, refers to the administration of the different active agents at different time points. The administration of the different active agents may overlap in time or may be temporally distinct. The cycles may encompass periods of time which are identical or which differ in length. For instance, the cycles may involve administration of the CpG nucleic acid on a daily basis, every two days, every three days, every four days, every five days, every six days, a weekly basis, a monthly basis or any set number of days or weeks there-between, every two months, three months, four months, five months, six months, seven months, eight months, nine months, ten months, eleven months, twelve months, etc, with the anti-HIV therapy being administered in between. Alternatively, the cycles may involve administration of the CpG nucleic acid on a daily basis for the first week, followed by a monthly basis for several months, and then every three months after that, with the anti-HIV therapy being administered in between. Any particular combination would be covered by the cycle schedule as long as it is determined that the appropriate schedule involves administration on a certain day.

### Certain Unmethylated CpG Containing Oligos Have B Cell Stimulatory Activity As Shown in vitro and in vivo

In the course of investigating the lymphocyte stimulatory effects of two antisense oligonucleotides specific for endogenous retroviral sequences, using protocols described in the attached Examples 1 and 2, it was surprisingly found that two out of twenty-four “controls” (including various scrambled, sense, and mismatch controls for a panel of “antisense” ODN) also mediated B cell activation and IgM secretion, while the other “controls” had no effect.

Two observations suggested that the mechanism of this B cell activation by the “control” ODN may not involve antisense effects 1) comparison of vertebrate DNA sequences listed in GenBank showed no greater homology than that seen with non-stimulatory ODN and 2) the two controls showed no hybridization to Northern blots with 10 μg of spleen poly A+RNA. Resynthesis of these ODN on a different synthesizer or extensive purification by polyacrylamide gel electrophoresis or high pressure liquid chromatography gave identical stimulation, eliminating the possibility of an impurity. Similar stimulation was seen using B cells from C3H/HeJ mice, eliminating the possibility that lipopolysaccharide (LPS) contamination could account for the results.

The fact that two “control” ODN caused B cell activation similar to that of the two “antisense” ODN raised the possibility that all four ODN were stimulating B cells through some non-antisense mechanism involving a sequence motif that was absent in all of the other nonstimulatory control ODN. In comparing these sequences, it was discovered that all of the four stimulatory ODN contained CpG dinucleotides that were in a different sequence context from the nonstimulatory control.

To determine whether the CpG motif present in the stimulatory ODN was responsible for the observed stimulation, over 300 ODN ranging in length from 5 to 42 bases that contained methylated, unmethylated, or no CpG dinucleotides in various sequence contexts were synthesized. These ODNs, including the two original “controls” (ODN 1 and 2) and two originally synthesized as “antisense” (ODN 3D and 3M; Krieg, A. M. J. Immunol. 143:2448 (1989)), were then examined for in vitro effects on spleen cells (representative sequences are listed in Table 1). Several ODN that contained CpG dinucleotides induced B cell activation and IgM secretion; the magnitude of this stimulation typically could be increased by adding more CpG dinucleotides (Table 1; compare ODN 2 to 2a or 3D to 3Da and 3Db). Stimulation did not appear to result from an antisense mechanism or impurity. ODN caused no detectable proliferation of .gamma..delta. or other T cell populations.

Mitogenic ODN sequences uniformly became nonstimulatory if the CpG dinucleotide was mutated (Table 1; compare ODN 1 to 1a; 3D to 3Dc; 3M to 3Ma; and 4 to 4a) or if the cytosine of the CpG dinucleotide was replaced by 5-methylcytosine (Table 1; ODN 1b,2b,2c,3Dd, and 3Mb). In contrast, methylation of other cytosines did not reduce ODN activity (ODN 1c, 2d, 3De and 3Mc). These data confirmed that a CpG motif is the essential element present in ODN that activate B cells.

In the course of these studies, it became clear that the bases flanking the CpG dinucleotide played an important role in determining the B cell activation induced by an ODN. The optimal stimulatory motif was determined to consist of a CpG flanked by two 5′ purines (preferably a GpA dinucleotide) and two 3′ pyrimidines (preferably a TpT or TpC dinucleotide). Mutations of ODN to bring the CpG motif closer to this ideal improved stimulation (e.g. compare ODN 2 to 2e; 3M to 3Md) while mutations that disturbed the motif reduced stimulation (e.g. compare ODN 3D to 3Df; 4 to 4b, 4c and 4d). On the other hand, mutations outside the CpG motif did not reduce stimulation (e.g. compare ODN 1 to 1d; 3D to 3Dg; 3M to 3Me).

Of those tested, ODNs shorter than 8 bases were non-stimulatory (e.g. ODN 4e). Among the forty-eight 8 base ODN tested, the most stimulatory sequence identified was TCAACGTT (ODN 4) which contains the self complementary “palindrome” AACGTT. In further optimizing this motif, it was found that ODN containing Gs at both ends showed increased stimulation, particularly if the ODN were rendered nuclease resistant by phosphorothioate modification of the terminal internucleotide linkages. ODN 1585 (5′ GGGGTCAACGTTCAGGGGGG 3′ (SEQ ID NO:1)), in which the first two and last five internucleotide linkages are phosphorothioate modified caused an average 25.4 fold increase in mouse spleen cell proliferation compared to an average 3.2 fold increase in proliferation induced by ODN 1638, which has the same sequence as ODN 1585 except that the 10 Gs at the two ends are replaced by 10 As. The effect of the G-rich ends is cis; addition of an ODN with poly G ends but no CpG motif to cells along with 1638 gave no increased proliferation.

Other octamer ODN containing a 6 base palindrome with a TpC dinucleotide at the 5′ end were also active if they were close to the optimal motif (e.g. ODN 4b,4c). Other dinucleotides at the 5′ end gave reduced stimulation (e.g. ODN 4f, all sixteen possible dinucleotides were tested). The presence of a 3′ dinucleotide was insufficient to compensate for the lack of a 5′ dinucleotide (eg. ODN 4g). Disruption of the palindrome eliminated stimulation in octamer ODN (eg., ODN 4h), but palindromes were not required in longer ODN.

TABLE 1

Oligonucleotide Stimulation of B Cells

Stimulation Index

| | |
|---|---|
| Sequence (5′ to 3′) | ³H Uridine IgM |

| | | |
|---|---|---|
| ODN | .dagger. | Production |

| | | |
|---|---|---|
| 1 (SEQ ID NO:2) | GCTAGACGTTAGCGT | 6.1 .+−. 0.8 17.9 .+−. 3.6 |
| 1a (SEQ ID NO:3) | ......T...... . | 1.2 .+−. 0.2 1.7 .+−. 0.5 |
| 1b (SEQ ID NO:4) | ......Z........ | 1.2 .+−. 0.1 1.8 .+−. 0.0 |
| 1c (SEQ ID NO:5) | ............Z.. | 10.3 .+−. 4.4 9.5 .+−. 1.8 |
| 1d (SEQ ID 140:6) | ..AT... ..GAGC. | 13.0 .+−. 2.3 18.3 .+−. 7.5 |
| 2 (SEQ ID NO:7) | ATGGAAGGTCCAGCGTTCTC | 2.9 .+−. 0.2 13.6 .+−. 2.0 |
| 2a (SEQ ID NO:8) | ..C..CTC..G......... | 7.7 .+−. 0.8 24.2 .+−. 3.2 |
| 2b (SEQ ID NO:9) | ..Z..CTC.ZG..Z...... | 1.6 .+−. 0.5 2.8 .+−. 2.2 |
| 2c (SEQ 10 NO:10) | ..Z..CTC..G......... | 3.1 .+−. 0.6 7.3 .+−. 1.4 |
| 2d (SEQ ID NO:11) | ..C..CTC..G......Z.. | 7.4 .+−. 1.4 27.7 .+−. 5.4 |
| 2e (SEQ ID NO:12) | ............A....... | 5.6 .+−. 2.0 ND |
| 3D (SEQ ID NO:13) | GAGAACGCTGGACCTTCCAT | 4.9 .+−. 0.5 19.9 .+−. 3.6 |
| 3Da (SEQ ID NO:14) | .........C.......... | 6.6 .+−. 1.5 33.9 .+−. 6.8 |
| 3Db (SEQ 10 NO:15) | .........C.......G.. | 10.1 .+−. 2.8 25.4 .+−. 0.8 |
| 3Dc (SEQ ID NO:16) | ...C.A.............. | 1.0 .+−. 0.1 1.2 .+−. 0.5 |
| 3Dd (SEQ ID NO:17) | .....Z.............. | 1.2 .+−. 0.2 1.0 .+−. 0.4 |
| 3De (SEQ ID NO:18) | .............Z...... | 4.4 .+−. 1.2 18.8 .+−. 4.4 |
| 3Df (SEQ ID NO:19) | ........A............ | 1.6 .+−. 0.1 7.7 .+−. 0.4 |
| 3Dg (SEQ ID NO:20) | ..... ....CC.G.ACTG.. | 6.1 .+−. 1.5 18.6 .+−. 1.5 |
| 3M (SEQ ID NO:21) | TCCATGTCGGTCCTGATGCT | 4.1 .+−. 0.2 23.2 .+−. 4.9 |
| 3Ma (SEQ ID NO:22) | ......CT............ | 0.9 .+−. 0.1 1.8 .+−. 0.5 |
| 3Mb (SEQ ID NO:23) | .......Z............ | 1.3 .+−. 0.3 1.5 .+−. 0.6 |
| 3Mc (SEQ ID NO:24) | ...........Z........ | 5.4 .+−. 1.5 8.5 .+−. 2.6 |
| 3Md (SEQ ID NO:25) | ......A..T.......... | 17.2 .4+−. 9.4 ND |
| 3Me (SEQ ID NO:26) | ...............C..A. | 3.6 .+−. 0.2 14.2 .+−. 5.2 |
| 4 | TCAACGTT | 6.1 .+−. 1.4 19.2 .+−. 5.2 |
| 4a | ....GC.. | 1.1 .+−. 0.2 1.5 .+−. 1.1 |
| 4b | ...GCGC. | 4.5 .+−. 0.2 9.6 .+−. 3.4 |
| 4c | ...TCGA. | 2.7 .4+−. 1.0 ND |
| 4d | ..TT..AA | 1.3 .4+−. 0.2 ND |
| 4e | −....... | 1.3 .+−. 0.2 1.1 .+−. 0.5 |
| 4f | C....... | 3.9 .+−. 1.4 ND |
| 4g | −−......CT | 1.4 .+−. 0.3 ND |
| 4h | .......C | 1.2 .+−. 0.2 ND |
| LPS | | 7.8 .+−. 2.5 4.8 .+−. 1.0 |

Stimulation indexes are the means and std. dev. derived from at least 3 separate experiments, and are compared to wells cultured with no added ODN. ND = not done. CpG dinucleotides are underlined. Dots indicate identity; dashes indicate deletions. Z indicates 5 methyl cytosine.)

The kinetics of lymphocyte activation were investigated using mouse spleen cells. When the cells were pulsed at the same time as ODN addition and harvested just four hours later, there was already a two-fold increase in ³H uridine incorporation. Stimulation peaked at 12-48 hours and then decreased. After 24 hours, no intact ODN were detected, perhaps accounting for the subsequent fall in stimulation when purified B cells with or without anti-IgM (at a submitogenic dose) were cultured with CpG ODN, proliferation was found to synergistically increase about 10-fold by the two mitogens in combination after 48 hours. The magnitude of stimulation was concentration dependent and consistently exceeded that of LPS under optimal conditions for both. Oligonucleotides containing a nuclease resistant phosphorothioate backbone were approximately two hundred times more potent than unmodified oligonucleotides.

Cell cycle analysis was used to determine the proportion of B cells activated by CpG-ODN. CpG-ODN induced cycling in more than 95% of B cells (Table 2). Splenic B lymphocytes sorted by flow cytometry into CD23− (marginal zone) and CD23+ (follicular) subpopulations were equally responsive to ODN-induced stimulation, as were both resting and activated populations of B cells isolated by fractionation over Percoll gradients. These studies demonstrated that CpG-ODN induce essentially all B cells to enter the cell cycle.

TABLE 2
Cell Cycle Analysis with CpG ODN Percent of cells in

| Treatment | G0 | G1 | SA + G2 + M |
|---|---|---|---|
| Media | 97.6 | 2.4 | 0.02 |
| ODN 1a | 95.2 | 4.8 | 0.04 |
| ODN 1d | 2.7 | 74.4 | 22.9 |
| ODN 3Db | 3.5 | 76.4 | 20.1 |
| LPS (30 μg/ml) | 17.3 | 70.5 | 12.2 |

The mitogenic effects of CpG ODN on human cells, were tested on peripheral blood mononuclear cells (PBMCs) obtained from two patients with chronic lymphocytic leukemia (CLL), as described in Example 1. Control ODN containing no CpG dinucleotide sequence showed no effect on the basal proliferation of 442 cpm and 874 cpm (proliferation measured by ³H thymidine incorporation) of the human cells. However, a phosphorothioate modified CpG ODN 3Md (SEQ ID NO: 25) induced increased proliferation of 7210 and 86795 cpm respectively in the two patients at a concentration of just 1 μM. Since these cells had been frozen, they may have been less responsive to the oligos than fresh cells in vivo. In addition, cells from CLL patients typically are non-proliferating, which is why traditional chemotherapy is not effective.

Certain B cell lines such as WEHI-231 are induced to undergo growth arrest and/or apoptosis in response to crosslinking of their antigen receptor by anti-IgM (Jakway, J. P. et al., “Growth regulation of the B lymphoma cell line WEHI-231 by anti-immunoglobulin, lipopolysaccharide and other bacterial products” J. Immunol. 137: 2225 (1986); Tsubata, T., J. Wu and T. Honjo: B-cell apoptosis induced by antigen receptor crosslinking is blocked by a T-cell signal through CD40.” Nature 364: 645 (1993)). WEHI-231 cells are rescued from this growth arrest by certain stimuli such as LPS and by the CD40 ligand. ODN containing the CpG motif were also found to protect WEHI-231 from anti-IgM induced growth arrest, indicating that accessory cell populations are not required for the effect.

To better understand the immune effects of unmethylated CpG ODN, the levels of cytokines and prostaglandins in vitro and in vivo were measured. Unlike LPS, CpG ODN were not found to induce purified macrophages to produce prostaglandin PGE2. In fact, no apparent direct effect of CpG ODN was detected on either macrophages or T cells. In vivo or in whole spleen cells, no significant increase in the following interleukins: IL-2, IL-3, IL-4, or IL-10 was detected within the first six hours. However, the level of IL-6 increased strikingly within 2 hours in the serum of mice injected with CpG ODN. Increased expression of IL-12 and interferon gamma (IFN-.gamma.) by spleen cells was also detected within the first two hours.

To determine whether CpG ODN can cause in vivo immune stimulation, DBA/2 mice were injected once intraperitoneally with PBS or phosphorothioate CpG or non-CpG ODN at a dose of 33 mg/kg (approximately 500 μg/mouse). Pharmacokinetic studies in mice indicate that this dose of phosphorothioate gives levels of approximately 10 μg/g in spleen tissue (within the effective concentration range determined from the in vitro studies described herein) for longer than twenty-four hours (Agrawal, S. et al. (1991) Proc. Natl. Acad. Sci. USA 91:7595). Spleen cells from mice were examined twenty-four hours after ODN injection for expression of B cells surface activation markers Ly-6A/E, Bla-1, and class II MHC using three color flow cytometry and for their spontaneous proliferation using ³H thymidine. Expression of all three activation markers was significantly increased in B cells from mice injected with CpG ODN, but not from mice injected with PBS or non-CpG ODN. Spontaneous ³H thymidine incorporation was increased by 2-6 fold in spleen cells from mice injected with the stimulatory ODN compared to PBS or non-CpG ODN-injected mice. After 4 days, serum IgM levels in mice injected with CpG ODN in vivo were increased by approximately 3-fold compared to controls. Consistent with the inability of these agents to activate T cells, there was minimal change in T cell expression of the IL-2R or CD-44.

Degradation of phosphodiester ODN in serum is predominantly mediated by 3′ exonucleases, while intracellular ODN degradation is more complex, involving 5′ and 3′ exonucleases and endonucleases. Using a panel of ODN bearing the 3D sequence with varying numbers of phosphorothioate modified linkages at the 5′ and 3′ ends, it was empirically determined that two 5′ and five 3′ modified linkages are required to provide optimal stimulation with this CpG ODN.

### Unmethylated CpG Containing Oligos Have NK Cell Stimulatory Activity

As described in further detail in Example 4, experiments were conducted to determine whether CpG containing oligonucleotides stimulated the activity of natural killer (NK) cells in addition to B cells. As shown in Table 3, a marked induction of NK activity among spleen cells cultured with CpG ODN 1 and 3Db was observed. In contrast, there was relatively no induction in effectors that had been treated with non-CpG control ODN.

TABLE 3
Induction Of NK Activity By CpG Oligodeoxynucleotides (ODN)

| | | | | % 2C11 Specific | |
|---|---|---|---|---|---|
| | % YAC-1 Specific Lysis* | | | Lysis | |
| | Effector:Target | | | Effector:Target | |
| ODN | 50:1 | | 100:1 | 50:1 | 100:1 |
| None | −1.1 | −1.4 | 15.3 | 16.6 | |
| 1 | 16.1 | | 24.5 | 38.7 | 47.2 |
| 3Db | 17.1 | | 27.0 | 37.0 | 40.0 |
| non-CpG ODN | −1.6 | −1.7 | 14.8 | 15.4 | |

### Neutralizing Activity of Methylated CpG Containing Oligos

B cell mitogenicity of ODN in which cytosines in CpG motifs or elsewhere were replaced by 5-methylcytosine were tested as described in Example 1. As shown in Table 1 above, ODN containing methylated CpG motifs were non-mitogenic (Table 1; ODN 1b, 2b, 3Db, and 3Mb). However, methylation of cytosines other than in a CpG dinucleotide retained their stimulatory properties (Table 1, ODN 1c, 2d, 3De, and 3Mc).

### Immunoinhibitory Activity of Oligos Containing a GCG Trinucleotide Sequence at or Near Both Termini

In some cases, ODN containing CpG dinucleotides that are not in the stimulatory motif described above were found to block the stimulatory effect of other mitogenic CpG ODN. Specifically the addition of an atypical CpG motif consisting of a GCG near or at the 5′ and/or 3′ end of CpG ODN actually inhibited stimulation of proliferation by other CpG motifs. Methylation or substitution of the cytosine in a GCG motif reverses this effect. By itself, a GCG motif in an ODN has a modest mitogenic effect, though far lower than that seen with the preferred CpG motif.

### Proposed Mechanisms of Action of Immunostimulatory, Neutralizing and Immunoinhibitory Oligonucleotides

Unlike antigens that trigger B cells through their surface Ig receptor, CpG-ODN did not induce any detectable Ca.sup.2+ flux, changes in protein tyrosine phosphorylation, or IP 3 generation. Flow cytometry with FITC-conjugated ODN with or without a CpG motif was performed as described in Zhao, Q et al.,(Antisense Research and Development 3:53-66 (1993)), and showed equivalent membrane binding, cellular uptake, efflux, and intracellular localization. This suggests that there may not be cell membrane proteins specific for CpG ODN. Rather than acting through the cell membrane, that data suggests that unmethylated CpG containing oligonucleotides require cell uptake for activity: ODN covalently linked to a solid Teflon support were nonstimulatory, as were biotinylated ODN immobilized on either avidin beads or avidin coated petri dishes. CpG ODN conjugated to either FITC or biotin retained full mitogenic properties, indicating no steric hindrance.

The optimal CpG motif (TGACGTT/C) is identical to the CRE (cyclic AMP response element). Like the mitogenic effects of CpG ODN, binding of CREB to the CRE is abolished if the central CpG is methylated. Electrophoretic mobility shift assays were used to determine whether CpG ODN, which are single stranded, could compete with the binding of B cell CREB/ATF proteins to their normal binding site, the doublestranded CRE. Competition assays demonstrated that single stranded ODN containing CpG motifs could completely compete the binding of CREB to its binding site, while ODN without CpG motifs could not. These data support the conclusion that CpG ODN exert their mitogenic effects through interacting with one or more B cell CREB/ATF proteins in some way. Conversely, the presence of GCG sequences or other atypical CpG motifs near the 5′ and/or 3′ ends of ODN likely interact with CREB/ATF proteins in a way that does not cause activation, and may even prevent it.

The stimulatory CpG motif is common in microbial genomic DNA, but quite rare in vertebrate DNA. In addition, bacterial DNA has been reported to induce B cell proliferation and immunoglobulin (Ig) production, while mammalian DNA does not (Messina, J. P. et al., J. Immunol. 147:1759 (1991)). Experiments further described in Example 3, in which methylation of bacterial DNA with CpG methylase was found to abolish mitogenicity, demonstrates that the difference in CpG status is the cause of B cell stimulation by bacterial DNA. This data supports the following conclusion: that unmethylated CpG dinucleotides present within bacterial DNA are responsible for the stimulatory effects of bacterial DNA.

Teleologically, it appears likely that lymphocyte activation by the CpG motif represents an immune defense mechanism that can thereby distinguish bacterial from host DNA. Host DNA would induce little or no lymphocyte activation due to it CpG suppression and methylation. Bacterial DNA would cause selective lymphocyte activation in infected tissues. Since the CpG pathway synergizes with B cell activation through the antigen receptor, B cells bearing antigen receptor specific for bacterial antigens would receive one activation signal through cell membrane Ig and a second signal from bacterial DNA, and would therefore tend to be preferentially activated. The interrelationship of this pathway with other pathways of B cell activation provide a physiologic mechanism employing a polyclonal antigen to induce antigen-specific responses.

### Method for Making Immunostimulatory Oligos

For use in the instant invention, oligonucleotides can be synthesized de novo using any of a number of procedures well known in the art. For example, the .beta.-cyanoethyl phosphoramidite method (S. L. Beaucage and M. H. Caruthers, (1981) Tet. Let. 22:1859); nucleoside H-phosphonate method (Garegg et al., (1986) Tet. Let. 27: 4051-4054; Froehler et al., (1986) Nucl. Acid. Res. 14: 5399-5407; Garegg et al., (1986) Tet. Let. 27: 4055-4058, is Gafffney et al., (1988) Tet. Let. 29:2619-2622). These chemistries can be performed by a variety of automated oligonucleotide synthesizers available in the market. Alternatively, oligonucleotides can be prepared from existing nucleic acid sequences (e.g. genomic or cDNA) using known techniques, such as those employing restriction enzymes, exonucleases or endonucleases.

For use in vivo, oligonucleotides are preferably relatively resistant to degradation (e.g. via endo- and exo-nucleases). Oligonucleotide stabilization can be accomplished via phosphate backbone modifications. A preferred stabilized oligonucleotide has a phosphorothioate modified backbone. The pharmacokinetics of phosphorothioate ODN show that they have a systemic half-life of forty-eight hours in rodents and suggest that they may be useful for in vivo applications (Agrawal, S. et al. (1991) Proc. Natl. Acad. Sci. USA 88:7595). Phosphorothioates may be synthesized using automated techniques employing either phosphoramidate or H phosphonate chemistries. Aryl- and alkyl-phosphonates can be made e.g. (as described in U.S. Pat. No. 4,469,863); and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described (Uhlmann, E. and Peyman, A. (1990) Chem. Rev. 90:544; Goodchild, J. (1990) Bioconjugate Chem. 1:165).

For administration in vivo, oligonucleotides may be associated with a molecule that results in higher affinity binding to target cell (e.g. B-cell and natural killer (NK) cell) surfaces and/or increased cellular uptake by target cells to form an “oligonucleotide delivery complex”. Oligonucleotides can be ionically, or covalently associated with appropriate molecules using techniques which are well known in the art. A variety of coupling or crosslinking agents can be used e.g. protein A, carbodiimide, and N-succinimidyl-3-(2-pyridyldithio) propionate (SPDP). Oligonucleotides can alternatively be encapsulated in liposomes or virosomes using well-known techniques.

### Therapeutic Uses of Immunostimulatory Oligos

Based on their immunostimulatory properties, oligonucleotides containing at least one unmethylated CpG dinucleotide can be administered to a subject in vivo to treat an “immune system deficiency”. Alternatively, oligonucleotides containing at least one unmethylated CpG dinucleotide can be contacted with lymphocytes (e.g. B cells or NK cells) obtained from a subject having an immune system deficiency ex vivo and activated lymphocytes can then be reimplanted in the subject.

Immunostimulatory oligonucleotides can also be administered to a subject in conjunction with a vaccine, as an adjuvant, to boost a subject's immune system to effect better response from the vaccine. Preferably the unmethylated CpG dinucleotide is administered slightly before or at the same time as the vaccine.

Preceding chemotherapy with an immunostimulatory oligonucleotide should prove useful for increasing the responsiveness of the malignant cells to subsequent chemotherapy. CpG ODN also increased natural killer cell activity in both human and murine cells. Induction of NK activity may likewise be beneficial in cancer immunotherapy.

The compositions of the invention may be delivered to a subject alone or in combination with a vector or a pharmaceutically acceptable carrier. In its broadest sense, a “vector” is any vehicle capable of facilitating the transfer of the compositions to the target cells. The vector generally transports the nucleic acid and/or the anti-HIV therapy to the target cells with reduced degradation relative to the extent of degradation that would result in the absence of the vector. Vectors also include oligonucleotide delivery complexes.

In general, the vectors useful in the invention are divided into two classes: biological vectors and chemical/physical vectors. Biological vectors and chemical/physical vectors are useful in the delivery and/or uptake of nucleic acids and anti-HIV therapy.

Biological vectors include, but are not limited to, plasmids, phagemids, viruses, other vehicles derived from viral or bacterial sources that have been manipulated by the insertion or incorporation of nucleic acid sequences, and free nucleic acid fragments which can be attached to nucleic acid sequences. Viral vectors are a preferred type of biological vector and include, but are not limited to, nucleic acid sequences from the following viruses: retroviruses, such as: Moloney murine leukemia virus; Harvey murine sarcoma virus; murine mammary tumor virus; Rous sarcoma virus; adenovirus; adeno-associated virus; SV40-type viruses; polyoma viruses; Epstein-Barr viruses; papilloma viruses; herpes viruses; vaccinia viruses; polio viruses; and RNA viruses such as any retrovirus. One can readily employ other viral vectors not named but known in the art.

Preferred viral vectors are based on non-cytopathic eukaryotic viruses in which non-essential genes have been replaced with a nucleic acid of interest (e.g., a nucleic acid encoding a cytokine such as IL-2). Non-cytopathic viruses include retroviruses, the life cycle of which involves reverse transcription of genomic viral RNA into DNA with subsequent proviral integration into host cellular DNA. Retroviruses have been approved for human gene therapy trials. In general, the retroviruses are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W. H. Freeman Co., N.Y. (1990) and Murry, E. J. Ed. “Methods in Molecular Biology,” vol. 7, Humana Press, Inc., Cliffton, N.J. (1991).

Another preferred virus for certain applications is the adeno-associated virus, a double-stranded DNA virus. The adeno-associated virus can be engineered to be replication-deficient and is capable of infecting a wide range of cell types and species. It further has advantages, such as heat and lipid solvent stability; high transduction frequencies in cells of diverse lineages; and lack of superinfection inhibition thus allowing multiple series of transductions. Reportedly, the adeno-associated virus can integrate into human insertional mutagenesis and variability of inserted gene expression. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other biological vectors include plasmid vectors. Plasmid vectors have been extensively described in the art and are well-known to those of skill in the art. See e.g., Sambrook et al., “Molecular Cloning: A Laboratory Manual,” Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operatively encoded within the plasmid. Some commonly used plasmids include pBR322, pUC 18, pUC19, pRC/CMV, SV40, and pBlueScript. Other plasmids are well-known to those of ordinary skill in the art. Additionally, plasmids may be custom designed using restriction enzymes and ligation reactions to remove and add specific fragments of DNA.

In addition to the biological vectors, chemical/physical vectors may be used to deliver a nucleic acid and/or an anti-HIV therapy to a target cell and facilitate uptake thereby. As used herein, a “chemical/physical vector” refers to a natural or synthetic molecule, other than those derived from bacteriological or viral sources, capable of delivering the nucleic acid and/or an anti-HIV therapy.

A preferred chemical/physical vector of the invention is a colloidal dispersion system. Colloidal dispersion systems include lipid-based systems including oil-in-water emulsions, micelles, mixed micelles, and liposomes. A preferred colloidal system of the invention is a liposome. Liposomes are artificial membrane vessels which are useful as a delivery vector in vivo or in vitro. It has been shown that large unilamellar vessels (LUV), which range in size from 0.2-4.0 μm can encapsulate large macromolecules. RNA, DNA and intact virions can be encapsulated within the aqueous interior and be delivered to cells in a biologically active form (Fraley, et al., Trends Biochem. Sci., (1981) 6:77).

Liposomes may be targeted to a particular tissue by coupling the liposome to a specific ligand such as a monoclonal antibody, sugar, glycolipid, or protein. Ligands which may be useful for targeting a liposome to an immune cell include, but are not limited to: intact or fragments of molecules which interact with immune cell specific receptors and molecules, such as antibodies, which interact with the cell surface markers of immune cells. Such ligands may easily be identified by binding assays well known to those of skill in the art. In still other embodiments, the liposome may be targeted to an infected cell by coupling it to an antibody that recognizes the cell. Additionally, the vector may be coupled to a nuclear targeting peptide, which will direct the vector to the nucleus of the host cell.

Lipid formulations for transfection are commercially available from QIAGEN, for example, as EFFECTENE™ (a non-liposomal lipid with a special DNA condensing enhancer) and SUPERFECT™ (a novel acting dendrimeric technology).

Liposomes are commercially available from Gibco BRL, for example, as LIPOFECTIN™ and LIPOFECTACE™, which are formed of cationic lipids such as N-[1-(2, 3 dioleyloxy)-propyl]-N, N, N-trimethylammonium chloride (DOTMA) and dimethyl dioctadecylammonium bromide (DDAB). Methods for making liposomes are well known in the art and have been described in many publications. Liposomes also have been reviewed by Gregoriadis, G. in Trends in Biotechnology, (1985) 3:235-241.

In one embodiment, the vehicle is a biocompatible microparticle or implant that is suitable for implantation or administration to the mammalian recipient. Exemplary bioerodible implants that are useful in accordance with this method are described in PCT International application no. PCT/US/03307 (Publication No. WO95/24929, entitled “Polymeric Gene Delivery System”. PCT/US/0307 describes a biocompatible, preferably biodegradable polymeric matrix for containing an exogenous gene under the control of an appropriate promoter. The polymeric matrix can be used to achieve sustained release of the CpG nucleic acid and/or the anti-HIV therapy in the subject.

The polymeric matrix preferably is in the form of a microparticle such as a microsphere (wherein the nucleic acid and/or the anti-HIV therapy is dispersed throughout a solid polymeric matrix) or a microcapsule (wherein the nucleic acid and/or anti-HIV therapy is stored in the core of a polymeric shell). Other forms of the polymeric matrix for containing the nucleic acid and/or the anti-HIV therapy include films, coatings, gels, implants, and stents. The size and composition of the polymeric matrix device is selected to result in favorable release kinetics in the tissue into which the matrix is introduced. The size of the polymeric matrix further is selected according to the method of delivery which is to be used, typically injection into a tissue or administration of a suspension by aerosol into the nasal and/or pulmonary areas. Preferably when an aerosol route is used the polymeric matrix and the nucleic acid and/or the anti-HIV therapy is encompassed in a surfactant vehicle. The polymeric matrix composition can be selected to have both favorable degradation rates and also to be formed of a material which is bioadhesive, to further increase the effectiveness of transfer when the matrix is administered to a nasal and/or pulmonary surface that has sustained an injury. The matrix composition also can be selected not to degrade, but rather, to release by diffusion over an extended period of time. In some preferred embodiments, the CpG nucleic acids are administered to the subject via an implant while the anti-HIV therapy is administered acutely.

In another embodiment the chemical/physical vector is a biocompatible microsphere that is suitable for delivery, such as oral or mucosal delivery. Such microspheres are disclosed in Chickering et al., Biotech. And Bioeng., (1996) 52:96-101 and Mathiowitz et al., Nature, (1997) 386:.410-414 and PCT Patent Application WO97/03702.

Both non-biodegradable and biodegradable polymeric matrices can be used to deliver the nucleic acid and/or the anti-HIV therapy to the subject. Biodegradable matrices are preferred. Such polymers may be natural or synthetic polymers. The polymer is selected based on the period of time over which release is desired, generally in the order of a few hours to a year or longer. Typically, release over a period ranging from between a few hours and three to twelve months is most desirable, particularly for the CpG nucleic acids. The polymer optionally is in the form of a hydrogel that can absorb up to about 90% of its weight in water and further, optionally is cross-linked with multi-valent ions or other polymers.

Bioadhesive polymers of particular interest include bioerodible hydrogels described by H. S. Sawhney, C. P. Pathak and J. A. Hubell in Macromolecules, (1993) 26:581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The CpG nucleic acid and the anti-HIV therapy may be administered alone (e.g. in saline or buffer) or using any delivery vectors known in the art. For instance the following delivery vehicles have been described: cochleates (Gould-Fogerite et al., 1994, 1996); Emulsomes (Vancott et al., 1998, Lowell et al., 1997); ISCOMs (Mowat et al., 1993, Carlsson et al., 1991, Hu et., 1998, Morein et al., 1999); liposomes (Childers et al., 1999, Michalek et al., 1989, 1992, de Haan 1995a, 1995b); live bacterial vectors (e.g., Salmonella, Escherichia coli, Bacillus calmatte-guerin, Shigella, Lactobacillus) (Hone et al., 1996, Pouwels et al., 1998, Chatfield et al., 1993, Stover et al., 1991, Nugent et al., 1998); live viral vectors (e.g., Vaccinia, adenovirus, Herpes Simplex) (Gallichan et al., 1993, 1995, Moss et al., 1996, Nugent et al., 1998, Flexner et al., 1988, Morrow et al., 1999); microspheres (Gupta et al., 1998, Jones et al., 1996, Maloy et al., 1994, Moore et al., 1995, O'Hagan et al., 1994, Eldridge et al., 1989); nucleic acid vaccines (Fynan et al., 1993, Kuklin et al., 1997, Sasaki et al., 1998, Okada et al., 1997, Ishii et al., 1997); polymers (e.g. carboxymethylcellulose, chitosan) (Hamajima et al., 1998, Jabbal-Gill et al., 1998); polymer rings (Wyatt et al., 1998); proteosomes (Vancott et al., 1998, Lowell et al., 1988, 1996, 1997); sodium fluoride (Hashi et al., 1998); transgenic plants (Tacket et al., 1998, Mason et al., 1998, Haq et al., 1995); virosomes (Gluck et al., 1992, Mengiardi et al., 1995, Cryz et al., 1998); and, virus-like particles (Jiang et al., 1999, Leibl et al., 1998).

The pharmaceutical compositions of the invention contain an effective amount of an CpG nucleic acid and optionally anti-HIV therapy and/or other therapeutic agents optionally included in a pharmaceutically-acceptable carrier. The term “pharmaceutically-acceptable carrier” means one or more compatible solid or liquid filler, dilutants or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The CpG nucleic acids and anti-HIV therapy may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Another suitable compound for sustained release delivery is GELFOAM, a commercially available product consisting of modified collagen fibers.

Alternatively, the active compounds may be in powder form for constitution with a suitable vehicle, e.g., sterile pyrogen-free water, before use.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

The CpG nucleic acid compositions and the anti-HIV therapy compositions can be administered on fixed schedules or in different temporal relationships to one another. The various combinations have many advantages over the prior art methods of treating HIV, particularly with regard to increased specific HIV toxicity and decreased non-specific toxicity to normal tissues.

Anti-HIV therapy and CpG nucleic acids can be administered by any ordinary route for administering medications. The anti-HIV therapy and the nucleic acids of the invention may be inhaled, ingested or administered by systemic routes. Systemic routes include oral and parenteral. Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers. Preferred routes of administration include but are not limited to oral, parenteral, intramuscular, intranasal, intratracheal, intrathecal, intravenous, inhalation, ocular, vaginal, and rectal.

For use in therapy, an effective amount of the CpG nucleic acid can be administered to a subject by any mode that delivers the nucleic acid to the affected organ or tissue, or alternatively to the immune system. “Administering” the pharmaceutical composition of the present invention may be accomplished by any means known to the skilled artisan. Preferred routes of administration include but are not limited to oral, parenteral, intramuscular, intranasal, intratracheal, inhalation, ocular, vaginal, and rectal.

For oral administration, the compounds (i.e., CpG nucleic acids, anti-HIV therapy, and the other therapeutic agent, such as adjuvants) can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. Techniques for preparing aerosol delivery systems are well known to those of skill in the art. Generally, such systems should utilize components which will not significantly impair the biological properties of the therapeutic, such as the immunostimulatory capacity of the nucleic acids (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing aerosols without resort to undue experimentation.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The present invention is further illustrated by the following Examples which in no way should be construed as further limiting. The entire contents of all of the references (including literature references, issued patents, published patent applications, and co-pending patent applications) cited throughout this application are hereby expressly incorporated by reference.

### EXAMPLES

### Example 1

### Effects of ODNs on B Cell Total RNA Synthesis and Cell Cycle

B cells were purified from spleens obtained from 6-12 wk old specific pathogen free DBA/2 or BXSB mice (bred in the University of Iowa animal care facility; no substantial strain differences were noted) that were depleted of T cells with anti-Thy-1.2 and complement and centrifugation over lympholyte M (Cedarlane Laboratories, Homby, Ontario, Canada) (“B cells”). B cells contained fewer than 1% CD4.sup.+ or CD8.sup.+ cells. 8×10.sup.4 B cells were dispensed in triplicate into 96 well microtiter plates in 100 μl RPMI containing 10% FBS (heat inactivated to 65° C. for 30 min.), 50 μM 2-mercaptoethanol, 100 U/ml penicillin, 100 ug/ml streptomycin, and 2 mM L-glutamate. 20 μM ODN were added at the start of culture for 20 h at 37° C., cells pulsed with 1 μCi of ³H uridine, and harvested and counted 4 hr later Ig secreting B cells were enumerated using the ELISA spot assay after culture of whole spleen cells with ODN at 20 μM for 48 hr. Data, reported in Table 1, represent the stimulation index compared to cells cultured without ODN. Cells cultured without ODN gave 687 cpm, while cells cultured with 20 μg/ml LPS (determined by titration to be the optimal concentration) gave 99,699 cpm in this experiment. ³H thymidine incorporation assays showed similar results, but with some nonspecific inhibition by thymidine released from degraded ODN (Matson. S and A. M. Krieg (1992) Nonspecific suppression of ³H-thymidine incorporation by control oligonucleotides. Antisense Research and Development 2:325).

For cell cycle analysis, 2×10⁶B cells were cultured for 48 hr. in 2 ml tissue culture medium alone, or with 30 μg/ml LPS or with the indicated phosphorothioate modified ODN at 1 μM. Cell cycle analysis was performed as described in (Darzynkiewicz, Z. et al., Proc. Natl. Acad. Sci. USA 78:2881 (1981)).

To test the mitogenic effects of CpG ODN on human cells, perpheral blood monocyte cells (PBMCs) were obtained from two patients with chronic lymphocytic leukemia (CLL), a disease in which the circulating cells are malignant B cells. Cells were cultured for 48 hrs and pulsed for 4 hours with tritiated thymidine as described above.

### Example 2

### Effects of ODN on Production of IgM from B Cells

Single cell suspensions from the spleens of freshly killed mice were treated with anti-Thyl, anti-CD4, and anti-CD8 and complement by the method of Leibson et al., J. Exp. Med. 154:1681 (1981)). Resting B cells (<,02% T cell contamination) were isolated from the 63-70% band of a discontinuous Percoll gradient by the procedure of DeFranco et al, J. Exp. Med. 155:1523 (1982). These were cultured as described above in 30 μM ODN or 20 μg/ml LPS for 48 hr. The number of B cells actively secreting IgM was maximal at this time point, as determined by ELIspot assay (Klinman, D. M. et al. J. Immunol. 144:506 (1990)). In that assay, B cells were incubated for 6 hrs on anti-Ig coated microtiter plates. The Ig they produced (>99% IgM) was detected using phosphatase-labelled anti-Ig (Southern Biotechnology Associated, Birmingham, Ala.). The antibodies produced by individual B cells were visualized by addition of BCIP (Sigma Chemical Co., St. Louis Mo.) which forms an insoluble blue precipitate in the presence of phosphatase. The dilution of cells producing 20-40 spots/well was used to determine the total number of antibody-secreting B cells/sample. All assays were performed in triplicate. In some experiments, culture supernatants were assayed for IgM by ELISA, and showed similar increases in response to CpG-ODN.

### Example 3

### B cell Stimulation by Bacterial DNA

DBA/2 B cells were cultured with no DNA or 50 μg/ml of a) Micrococcus lysodeikticus; b) NZB/N mouse spleen; and c) NFS/N mouse spleen genomic DNAs for 48 hours, then pulsed with ³H thymidine for 4 hours prior to cell harvest. Duplicate DNA samples were digested with DNAse I for 30 minutes at 37° C. prior to addition to cell cultures. E coli DNA also induced an 8.8 fold increase in the number of IgM secreting B cells by 48 hours using the ELISA-spot assay.

DBA/2 B cells were cultured with either no additive, 50 μg/ml LPS or the ODN 1; 1a; 4; or 4a at 20 uM. Cells were cultured and harvested at 4, 8, 24 and 48 hours. BXSB cells were cultured as in Example 1 with 5, 10, 20, 40 or 80 μM of ODN 1; 1a; 4; or 4a or LPS. In this experiment, wells with no ODN had 3833 cpm. Each experiment was performed at least three times with similar results. Standard deviations of the triplicate wells were <5%.

### Example 4

### Effects of ODN on Natural Killer (NK) Activity

10×10⁶ C57BL/6 spleen cells were cultured in two ml RPMI (supplemented as described for Example 1) with or without 40 μM CpG or non-CpG ODN for forty-eight hours. Cells were washed, and then used as effector cells in a short term ⁵¹Cr release assay with YAC-1 and 2C11, two NK sensitive target cell lines (Ballas, Z. K. et al. (1993) J. Immunol. 150:17). Effector cells were added at various concentrations to 10^{4 51}Cr-labeled target cells in V-bottom microtiter plates in 0.2 ml, and incubated in 5% CO.sub.2 for 4 hr. at 37° C. Plates were then centrifuged, and an aliquot of the supernatant counted for radioactivity. Percent specific lysis was determined by calculating the ratio of the ⁵¹Cr released in the presence of effector cells minus the ⁵¹Cr released when the target cells are cultured alone, over the total counts released after cell lysis in 2% acetic acid minus the ⁵¹Cr cpm released when the cells are cultured alone.

### Example 5

### In vivo Studies With CpG Phosphorothioate ODN

Mice were weighed and injected IP with 0.25 ml of sterile PBS or the indicated phophorothioate ODN dissolved in PBS. Twenty four hours later, spleen cells were harvested, washed, and stained for flow cytometry using phycoerythrin conjugated 6B2 to gate on B cells in conjunction with biotin conjugated anti Ly-6A/E or anti-Ia^{d} (Pharmingen, San Diego, Calif.) or anti-Bla-1 (Hardy, R. R. et al., J. Exp. Med. 159:1169 (1984). Two mice were studied for each condition and analyzed individually.

### Example 6

### Titration of Phosphorothioate ODN for B Cell Stimulation

B cells were cultured with phosphorothioate ODN with the sequence of control ODN 1a or the CpG ODN 1d and 3Db and then either pulsed after 20 hr with ³H uridine or after 44 hr with ³H thymidine before harvesting and determining cpm.

### Example 7

### Rescue of B Cells From Apoptosis

WEHI-231 cells (5×10⁴/well) were cultured for 1 hr. at 37° C. in the presence or absence of LPS or the control ODN 1a or the CpG ODN 1d and 3Db before addition of anti-IgM (1 μ/ml). Cells were cultured for a further 20 hr. before a 4 hr. pulse with 2 μCi/well ³H thymidine. In this experiment, cells with no ODN or anti-IgM gave 90.4.×10³ by addition of anti-IgM. The phosphodiester ODN shown in Table 1 gave similar protection, though with some nonspecific suppression due to ODN degradation. Each experiment was repeated at least 3 times with similar results.

### Example 8

### In vivo Induction of IL-6

DBA/2 female mice (2 mos. old) were injected IP with 500 μg CpG or control phosphorothioate ODN. At various time points after injection, the mice were bled. Two mice were studied for each time point. IL-6 was measured by ELISA, and IL-6 concentration was calculated by comparison to a standard curve generated using recombinant IL-6. The sensitivity of the assay was 10 pg/ml. Levels were undetectable after 8 hr.

### Example 9

Binding of B cell CREB/ATF to a Radiolabelled Doublestranded CRE Probe (CREB)

Whole cell extracts from CH12.LX B cells showed 2 retarded bands when analyzed by EMSA with the CRE probe (free probe is off the bottom of the figure). The CREB/ATF protein(s) binding to the CRE were competed by the indicated amount of cold CRE, and by single-stranded CpG ODN, but not by non-CpG ODN.

### Equivalents

Those skilled in the art will recognize, or be able to ascertain using no more than routine experimentation, many equivalents of the specific embodiments of the invention described herein. Such equivalents are intended to be encompassed by the following claims.

All references, patents and patent publications that are recited in this application are incorporated in their entirety herein by reference.

1. A method for treating a subject, comprising: administering a CpG nucleic acid to a subject infected with human immunodeficiency virus (HIV) in an effective amount to treat HIV infection.

2. The method of claim 1, wherein the CpG nucleic acid does not include a palindrome.

3. The method of claim 1, wherein the CpG nucleic acid is an adjuvant-type CpG nucleic acid.

4. The method of claim 1, wherein the CpG nucleic acid is a IFN-α-inducing CpG nucleic acid.

5. The method of claim 1, further comprising administering an anti-HIV therapy.

6. The method of claim 5, wherein the anti-HIV therapy is an inhibitor of HIV replication.

7. The method of claim 6, wherein the inhibitor of HIV replication is a protease inhibitor.

8. The method of claim 6, wherein the inhibitor of HIV replication is HAART.

9. The method of claim 5, wherein the anti-HIV therapy is a cytokine or a chemokine.

10. The method of claim 5, wherein the anti-HIV therapy is administered in a sub-therapeutic dosage and wherein the combination of the sub-therapeutic dose of the anti-HIV therapy and the CpG nucleic acid produce a therapeutic result in the treatment of HIV infection.

11. The method of claim 5, wherein the CpG nucleic acid is administered in a sub-therapeutic dosage and wherein the combination of the sub-therapeutic dose of the anti-HIV therapy and the CpG nucleic acid produce a therapeutic result in the treatment of HIV infection.

12. The method of claim 5, wherein the anti-HIV therapy is administered at the same time as the CpG nucleic acid.

13. The method of claim 5, wherein the anti-HIV therapy is administered prior to the CpG nucleic acid.

14. The method of claim 5, wherein the anti-HIV therapy is administered prior to the initial administration of CpG nucleic acid and the anti-HIV therapy is continued during the administration of the CpG nucleic acid.

15. The method of claim 14, wherein the anti-HIV therapy is terminated.

16. The method of claim 15, wherein the anti-HIV therapy is terminated at least one week after the initial administration of CpG.

17. The method of claim 5, wherein the CpG nucleic acid is administered prior to the initial administration of anti-HIV therapy and the CpG nucleic acid is continued during the administration of the anti-HIV therapy.

18. The method of claim 5, wherein the CpG nucleic acid and the anti-HIV therapy are administered in alternating cycles.

19. The method of claim 18, wherein the alternating cycles are monthly cycles.

20. The method of claim 9, wherein the cytokine is T-cell activating cytokine.

21. The method of claim 9, wherein the T-cell activating cytokine is IL-2.

22. The method of claim 9, wherein the chemokine is selected from the group consisting of RANTES and MIP-1α.

23. The method of claim 1, further comprising administering a non-steroidal anti-inflammatory agent.

24. The method of claim 23, wherein the non-steroidal anti-inflammatory agent is Piroxicam, Mefenamic acid, Nabumetone, Sulindac, Tolmetin, Ketorolac, Rofecoxib, Diclofenac, Naproxen, Flurbiprofen, Celecoxib, Oxaprozin, Diflunisal, Etodolac, Fenoprofen, Ibuprofen, Indomethacin, Ketoprofen, Etodolac, and Meloxicam.

25. The method of claim 3, wherein the adjuvant-type CpG nucleic acid has a sequence including at least the following formula:

| | |
|---|---|
| 5′ TCN₁TN₂X₁X₂CGTT]N₃[X₁X₂CGTT]N₄[X₁X₂CGTT] 3′, | (SEQ ID NO: 33) |

wherein N₄ is about 0-26 bases with the proviso that N₄ does not contain a CCGG quadmer or more than one CCG or CGG trimer.

26. The method of claim 25, wherein N₄ is selected from the group consisting of nothing, any nucleotide, C, T, TT, TTT, TTTT, and TC.

27. The method of claim 25, wherein N₃ and N₄ are both TT.

28. The method of claim 25, wherein X₂ is T.

29. The method of claim 25, wherein X₁ is G.

30. The method of claim 4, wherein the IFN-α-inducing CpG nucleic acid comprises the following sequence

| | |
|---|---|
| 5′ Y₁N₁X₁X₂CGX₃X₄N₂Y₂ 3′, | (SEQ ID NO: 73) |

wherein G is guanine; C is unmethylated cytosine; X₁, X₂, X₃, and X₄ independently are single nucleotides; N₁ and N₂ are independently nucleic acid molecules each having between 0 and 20 nucleotides; N₁X₁X₂CGX₃X₄N₂ (SEQ ID NO: 74) includes a palindrome at least 6 nucleotides long that contains at least one CG; Y₁ is a nucleic acid molecule having between 1 and 8 nucleotides comprising at least one modified internucleotide linkage; and Y₂ is independently a nucleic acid molecule having between 3 and 8 nucleotides comprising at least 3 consecutive Gs and at least one modified internucleotide linkage.

31. The method of claim 30, wherein at least one modified internucleotide linkage is a phosphorothioate modified linkage.

32. The method of claim 30, wherein Y₁ is comprised of at least 3 Gs.

33. The method of claim 30, wherein Y₁ is comprised of all Gs.

34. The method of claim 30, wherein Y₂ is comprised of at least 4 Gs.

35. The method of claim 30, wherein Y₂ is comprised of all Gs.

36. The method of claim 30, wherein Y₁ includes between two and five modified internucleotide linkages and Y₂ includes between two and five modified internucleotide linkages.

37. The method of claim 30, wherein the palindrome has a phosphodiester backbone.

38. The method of claim 1, wherein the CpG nucleic acid has less than or equal to 100 nucleotides.

39-53. (Canceled)

54. A method for treating a subject, comprising: administering a CpG nucleic acid and an anti-HIV therapy to a subject infected with human immunodeficiency virus (HIV) in an effective amount to treat HIV infection.

55-86. (Canceled)
